(19) United States
(12) Patent Application Publication
(10) Pub. No.: US 2022/0135651 A1
(43) Pub. Date: May 5, 2022

(54) A MEMBRANE-BOUND FIT-1 DECOY AND USES THEREOF

(71) Applicant: AGENCY FOR SCIENCE, TECHNOLOGY AND RESEARCH, Connexis (SG)

(72) Inventors: Yiqi SEOW, Proteos (SG); Ming Li Lalita LAU, Proteos (SG)

(21) Appl. No.: 17/430,037

(22) PCT Filed: Feb. 17, 2020
(86) PCT No.: PCT/SG2020/050080

§ 371 Date: Aug. 11, 2021

(30) Foreign Application Priority Data
Feb. 18, 2019 (SG) 10201901383S

(51) Int. Cl. C07K 14/71; C12N 15/113

(52) U.S. Cl. CPC C07K 14/71; C12N 15/1136; A61K 38/00; C12N 2310/141; C07K 2319/03

(57) ABSTRACT

We describe a Flt-1 decoy comprising a VEGF binding domain of Flt-1 and a membrane anchoring domain, in which the Flt-1 decoy does not substantially comprise an intracellular domain.

### CROSS-REFERENCE TO RELATED PATENT APPLICATIONS

This application is a U.S. National Phase Application under 35 U.S.C. § 371 of International Patent Application No. PCT/SG2020/050080, filed Feb. 17, 2020, and claims priority to SG 10201901383S, filed Feb. 18, 2019, all of which are incorporated by reference in their entireties. The International Application was published on Aug. 27, 2020 as International Publication No. WO 2020/171777 A1.

### FIELD

This invention relates to the fields of medicine, cell biology, molecular biology and genetics. This invention relates to the field of medicine.

### BACKGROUND

Diseases characterized by aberrant neovascularization include wet age-related macular degeneration (nAMD) and cancers such as glioblastoma and colon cancers, can be treated by therapies inhibiting vascular endothelial growth factor (VEGF).

These include antibody-based inhibition (Avastin & Lucentis), aptamer (Macugen) and soluble decoys based on the VEGF receptor Flt-1 (Eyelea).

The worldwide market for anti-VEGF therapeutics to tackle aberrant neovascularization is huge and competitive, hence the considerable interest in tackling this issue. For example, with AMD affects 0.4% of people between 50 and 60; 0.7% between 60 and 70; 2.3% between 70-80 and 12% older than 80, of which up to 20% has wet-AMD which is amenable to anti-VEGFs, making the market extremely lucrative for the aging population in the developed world. Avastin, an anti-VEGF single chain antibody, is also used for colon cancer, lung cancer, glioblastoma and renal-cell carcinoma.

Current protein and antibody-based therapy has proven to be effective in the stabilisation and improvement of vision in wet AMD and inhibition of cancer proliferation.

There are however a number of problems with current treatments. The treatment posology of protein and antibody-based therapy dictates that frequent injections of anti-VEGF agents are required, over many years, to maintain vision and to control the disease process.

### SUMMARY

Current treatments come with a significant burden in that they require frequent administrations over lengthy periods of time.

We disclose a nucleotide sequence for a membrane-bound Flt-1 lacking the C-terminus intracellular signalling domain that has been codon-optimized for expression in mammalian cells.

We also disclose nucleotide sequences for mirtrons (RNAi effectors) targeting VEGF nested as introns within the coding region of above-mentioned Flt-1.

We describe a combinatorial therapy that includes (1) a membrane-bound Flt-1 decoy which has its intracellular signalling domain removed; and (2) mirtrons that target VEGF delivered as a genetic construct using AAV2 or AAV-DJ.

According to a 1^{st} aspect of the present invention, we provide an Flt-1 decoy. The Flt-1 decoy may comprise a VEGF binding domain of Flt-1. The Flt-1 decoy may comprise a membrane anchoring domain. The Flt-1 decoy may be such that it does not substantially comprise an intracellular domain.

The Flt-1 decoy may be such that it is not soluble.

The Flt-1 decoy may be such that it is not capable of signal transduction. It may be incapable of signalling through an SHC-GRB2, PI3K or PLC pathway.

The Flt-1 decoy may lack an intracellular domain. The Flt-1 decoy may lack an intracellular signalling domain of Flt-1. The Flt-1 decoy may lack amino acids 819 to 1338 of GenBank Accession Number: NP 002010.2.

The Flt-1 decoy may be such that the VEGF binding domain of Flt-1 comprises a sequence comprising amino acids 27 to 250 of GenBank Accession Number: NP_002010.2. The Flt-1 decoy may comprise a fragment, homologue, variant or derivative thereof. The fragment, homologue, variant or derivative thereof may be capable of binding VEGF, preferably VEGF-A (NP_001020537.2) or VEGF-B (NP_001230662.1).

The Flt-1 decoy may be such that the membrane anchoring domain comprises a transmembrane domain of Flt1 comprising amino acids 759 to 780 of GenBank Accession Number: NP_002010.2. The Flt-1 decoy may comprise a fragment, homologue, variant or derivative thereof capable of anchoring the Flt-1 decoy to a cell membrane.

The Flt-1 decoy may comprise the sequence of Flt-1 (GenBank Accession Number: NM_002019.4) but without the intracellular domain (amino acids 819 to 1338 of GenBank Accession Number: NP_002010.2). The Flt-1 decoy may comprise a fragment, homologue, variant or derivative thereof which is capable of binding VEGF and which is not soluble.

There is provided, according to a 2^{nd} aspect of the present invention, a combination of a Flt-1 decoy according to the 1ₛₜ aspect of the invention together with a mirtron capable of inhibiting VEGF.

The mirtron may comprise

(SEQ ID NO: 1)
GTAAATGTATGTATGTGGGTGTTCAAGAGACACCCACACACATACATC
TCAGTTTTTTCTCTTTCTTTCAG

The mirtron may comprise

(SEQ ID NO: 2)
GTAGATTATGCGGATTAAATTTCAAGAGAGTTTGATCCGCATAATCTGT
CAGTTTTTTCTCTTTCTTTCAG

We provide, according to a 3^{rd} aspect of the present invention, use of a Flt-1 decoy or a combination as set out above in the preparation of a medicament for use as a VEGF inhibitor.

The Flt-1 decoy or combination may be provided for use in a method of treatment or prevention of a disease.

The disease may comprise macular degeneration, such as age related macular degeneration (AMD) or wet AMD, corneal neovascularization, diabetic retinopathy, retinal vein occlusions, retinopathy of prematurity, or any other ocular disease presenting with neovascularization. The disease may comprise cancer such as colon cancer, lung cancer, breast cancer, gastrointestinal stromal cancer, hepatocellular carcinoma, ovarian cancer, fallopian tube cancer, cervical cancer, primary peritoneal cancer, thyroid cancer, pancreatic neuroendocrine tumour, soft tissue sarcoma, glioblastoma or renal-cell carcinoma.

As a 4^{th} aspect of the present invention, there is provided a nucleic acid capable of encoding an Flt-1 decoy or a combination as set out above.

We provide, according to a 5^{th} aspect of the present invention, an expression vector capable of encoding an Flt-1 decoy or a combination as set out above.

The expression vector may comprise a viral expression vector, adenoviral expression vector, adeno-associated expression vector, a plasmid construct naked or complexed with liposomes or polymersomes or a dumbbell DNA construct.

The practice of this invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1 is a diagram demonstrating the difference in Flt1, Vegf-Trap, coFlt1 and sFlt1.

FIG. 2 is a diagram showing that coFlt1 and coFlt1-Mirt are effective at VEGF sequestration and inhibition.

FIG. 2A is a conceptual diagram for the genetic constructs used in FIG. 2B to FIG. 2E.

FIG. 2B is a diagram showing anti-HA and anti-His Western blots of cell lysates from HEK293 cells transfected with pcoFlt1, pcoFlt1-Mirt and pscoFlt1 harvested 2 days after transfection.

FIG. 2C is a diagram showing quantification of VEGF by ELISA in HEK293 cells transfected with pcoFlt1, pcoFlt1-Mirt or pscoFlt1. 2 days after transfection, 2 ng/ml of Vegf added to the cell culture medium for 2 hours and the cell culture supernatant was removed to be assayed. As scoFlt1 is soluble and would thus be in the medium, it was removed by incubation with His-tag Dynabeads for 30 minutes. Then all supernatant was quantified for VEGF using ELISA.

FIG. 2D is a diagram showing relative luciferase expression level using a dual luciferase kit 2 days after co-transfection of psiCheck2.2 VegfT1 with pcoFlt1 and pcoFlt1-Mirt in HEK293 cells.

FIG. 2E is a diagram showing VEGF mRNA expression normalised to GAPDH 2 days after cobalt chloride addition.

### DETAILED DESCRIPTION

Gene therapy, whether mediated by AAV or other gene delivery modality, with its ability to provide longer, sustained protein expression, appears to be a promising alternative solution in which the patients would require only treatment once every 6 month or even more because the treatment is genetically encoded with a much longer therapeutic half-life than protein-based therapeutics.

Previous attempts have employed soluble Flt-1, which has a number of limitations:

Being soluble, it can leak out of the site of expression and have adverse effects which cannot be anticipated with the experience of short-lived injectable antibodies or protein

Flt-1 binds to VEGF as a dimer and as such, requires proper dimerization in order to bind VEGF.

In the membrane, the Flt-1 monomer has 3 degrees of freedom of movement which aids dimerization; soluble Flt-1 monomer has 6 degrees of freedom of movement which greatly reduces the chance of dimerization, necessitating higher doses to bind to the same amount of VEGF.

### Flt-1 Decoy

We therefore provide an Flt-1 decoy which solves the above problems.

The Flt-1 decoy described here comprises a VEGF binding portion, for example, an extracellular domain of Flt-1.

The Flt-1 decoy comprises a membrane anchoring portion which anchors the Flt-1 decoy to a plasma membrane. Our Flt-1 is therefore capable of binding VEGF but is bound to the plasma membrane and is not soluble. Given this, lower doses of Flt-1 decoy are required to bind to dimerize and bind to Flt-1.

The Flt-1 decoy described in this document further lacks an intracellular domain. The Flt-1 decoy therefore does not have any intracellular signalling function.

A Flt-1 decoy as described in this document may be made by combining any of the components set out above. Details of such methods of construction are set out below.

### VEGF

As used in this document, VEGF refers to an polypeptide having an amino acid sequence:

GenBank Accession Number NP_001020537.2
(SEQ ID NO: 3)
MTDRQTDTAPSPSYHLLPGRRRTVDAAASRGQGPEPAPGGGVEGVGARG
VALKLFVQLLGCSRFGGAVVRAGEAEPSGAARSASSGREEPQPEEGEEE
EEKEEERGPQWRLGARKPGSWTGEAAVCADSAPAARAPQALARASGRGG
RVARRGAEESGPPHSPSRRGSASRAGPGRASETMNFLLSWVHWSLALLL
YLHHAKWSQAAPMAEGGGQNHHEVVKFMDVYQRSYCHPIETLVDIFQEY
PDEIEYIFKPSCVPLMRCGGCCNDEGLECVPTEESNITMQIMRIKPHQG
QHIGEMSFLQHNKCECRPKKDRARQEKKSVRGKGKGQKRKRKKSRYKSW
SVYVGARCCLMPWSLPGPHPCGPCSERRKHLFVQDPQTCKCSCKNTDSR
CKARQLELNERTCRCDKPRR

Fragments, homologues, variants and derivatives (as described below) of such a sequence having a VEGF activity, such as inducing angiogenesis and promoting proliferation.

Assays for such activities are known in the art. For example, an assay for angiogenesis activity may comprise a HUVEC angiogenic assay. By low-density plating HUVECs on matrigel with or without angiogenic agent, the ability of HUVEC to form matrices may be assayed. An assay for promoting proliferation may be based on cell counts with or without VEGF equivalent.

Vascular endothelial growth factor (VEGF) is a potent angiogenic factor and was first described as an essential growth factor for vascular endothelial cells. VEGF is up-regulated in many tumours and its contribution to tumor angiogenesis is well defined. In addition to endothelial cells, VEGF and VEGF receptors are expressed on numerous non-endothelial cells including tumor cells.

VEGF is described in detail in for example Duffy A M, Bouchier-Hayes D J, Harmey J H. Vascular Endothelial Growth Factor (VEGF) and Its Role in Non-Endothelial Cells: Autocrine Signalling by VEGF. In: Madame Curie Bioscience Database [Internet]. Austin (Tex.): Landes Bioscience; 2000-2013. Available from: https://www.ncbi.nlm nih.gov/books/NBK6482/

### VEGF Binding Domain

In order to bind to VEGF, the Flt-1 decoy described in this document comprises a portion capable of binding VEGF.

The VEGF binding portion may comprise a VEGF binding domain, such as a VEGF binding domain of Flt-1.

The VEGF binding domain of Flt-1 may comprise an extracellular portion or domain of Flt-1.

For example, the VEGF binding domain of Flt-1 may comprise the following amino acid sequence:

GenBank Accession Number NP_002010.2 1-250
(SEQ ID NO: 4)
MVSYWDTGVLLCALLSCLLLTGSSSGSKLKDPELSLKGTQHIMQAGQTL
HLQCRGEAAHKWSLPEMVSKESERLSITKSACGRNGKQFCSTLTLNTAQ
ANHTGFYSCKYLAVPTSKKKETESAIYIFISDTGRPFVEMYSEIPEIIH
MTEGRELVIPCRVTSPNITVTLKKFPLDTLIPDGKRIIWDSRKGFIISN
ATYKEIGLLTCEATVNGHLYKTNYLTHRQTNTIIDVQISTPRPVKLLRG
HTLVL

Fragment, homologues, variants and derivatives of such a sequence, as described below, may be employed. Such fragments, homologues, variants and derivatives may be capable of binding specifically to VEGF. Assays for VEGF binding are known in the art and are described, for example, in Park et al (1994). Placenta growth factor. Potentiation of vascular endothelial growth factor bioactivity, in vitro and in vivo, and high affinity binding to Flt-1 but not to Flk-1/KDR. J Biol Chem. 1994 Oct. 14; 269(41):25646-54).

### Membrane Anchoring Domain

The Flt-1 decoy described in this document may further comprise a portion capable of anchoring the Flt-1 decoy to a membrane, such as a plasma or cell membrane.

The presence of the membrane anchoring portion ensures that the Flt-1 decoy is bound to the membrane, instead of being in solution. Binding to a membrane restricts the degrees of freedom of the Flt-1 and encourages dimerization of the Flt-1 decoy.

The membrane anchoring portion may comprise a transmembrane domain.

The transmembrane domain may be from any polypeptide, for example amino acids 765 to 785 of GenBank Accession Number: NP_002244.1 or amino acids 376 to 399 of GenBank Accession Number: NP_002285. For example, the Flt-1 decoy may comprise a transmembrane domain of Flt-1 having the sequence:

GenBank Accession Number NP_002010.2
(residues 759-780)
(SEQ ID NO: 5)
LITLTCTCVAATLFWLLLTLFI

Fragments, homologues, variants and derivatives (as described below) of such a sequence having domain anchoring activity, are also included.

### Intracellular Domain of Flt-1

The Flt-1 decoy as described in this document does not substantially comprise an intracellular or domain. That is to say, the Flt-1 decoy may be such that it does not substantially include any portion that is within the cell.

The intracellular domain of Flt-1 is responsible for signalling. The Flt-1 decoy may therefore lack any intracellular signalling function. It may be incapable of signalling through an SHC-GRB2, PI3K or PLC pathway for example.

The intracellular or cytoplasmic domain of Flt-1 comprises amino acids 781 to 1338 of GenBank Accession Number: NP 002010.2.

Within this portion of the intracellular domain, Flt-1 contains a number of residues which are responsible for its signalling activities. These comprise phosphorylation and binding sites, for example.

The Flt-1 decoy may lack any one or more, or all of the intracellular sequence containing these sites.

The Flt-1 decoy may lack a portion of the intracellular domain containing a signalling site. It may lack a portion of the intracellular domain containing a phosphorylation site.

It will be appreciated that the Flt-1 decoy may comprise a portion of the intracellular sequence of Flt-1, so long that portion does not comprise one or more, preferably all, of the residues involved in signalling.

The Flt-1 decoy may comprise a portion of the intracellular signalling sequence so long as it does not contain a signalling site. It may comprise a portion of the intracellular signalling sequence so long as it does not contain a phosphorylation site.

A skilled person will understand that such Flt-1 decoys are within the meaning of the term “substantially comprising an intracellular domain” and “does not substantially include any portion that is within the cell”.

Flt-1 Signalling Residues

Residues in the intracellular domain of Flt-1 which are involved in signalling are known in the art. Such residues include the following, with reference to the sequence in GenBank Accession Number: NP 002010.2 and with activities associated with the residue set out in parentheses:
861K (enzyme activity, interaction with PLCG)
914Y (phosphorylation at other tyrosine residues)
1050N (kinase activity, activity in promoting proliferation of endothelial cells)
1169Y (phosphorylation site, interaction with PLCG)
1213Y (phosphorylation site, interaction with PIK3R1)
1242Y (phosphorylation site)
1327Y (phosphorylation site)
1333Y (phosphorylation site, abolishes interaction with CBL)

The Flt-1 decoy may therefore lack any portion of the intracellular domain of Flt-1 which includes any one or more, such as all, of these residues.

For example, the Flt-1 decoy may lack the whole of the intracellular domain. Such a Flt-1 decoy lacks a sequence corresponding to amino acids 781 to 1338 of GenBank Accession Number: NP 002010.2.

The Flt-1 decoy may lack an intracellular domain sequence which has a phosphorylation site, i.e., the Flt-1 decoy lacks an intracellular phosphorylation site.

Such a Flt-1 decoy may lack a sequence corresponding to positions 781 to 1338, positions 782 to 1338, positions 783 to 1338, positions 784 to 1338, positions 785 to 1338, positions 786 to 1338, positions 787 to 1338, positions 788 to 1338, positions 789 to 1338, positions 790 to 1338, positions 791 to 1338, positions 792 to 1338, positions 793 to 1338, positions 794 to 1338, positions 795 to 1338, positions 796 to 1338, positions 797 to 1338, positions 798 to 1338, positions 799 to 1338, positions 800 to 1338, positions 801 to 1338, positions 802 to 1338, positions 803 to 1338, positions 804 to 1338, positions 805 to 1338, positions 806 to 1338, positions 807 to 1338, positions 808 to 1338, positions 809 to 1338, positions 810 to 1338, positions 811 to 1338, positions 812 to 1338, positions 813 to 1338, positions 814 to 1338, positions 815 to 1338, positions 816 to 1338, positions 817 to 1338, positions 818 to 1338, positions 819 to 1338, positions 820 to 1338, positions 821 to 1338, positions 822 to 1338, positions 823 to 1338, positions 824 to 1338, positions 825 to 1338, positions 826 to 1338, positions 827 to 1338, positions 828 to 1338, positions 829 to 1338, positions 830 to 1338, positions 831 to 1338, positions 832 to 1338, positions 833 to 1338, positions 834 to 1338, positions 835 to 1338, positions 836 to 1338, positions 837 to 1338, positions 838 to 1338, positions 839 to 1338, positions 840 to 1338, positions 841 to 1338, positions 842 to 1338, positions 843 to 1338, positions 844 to 1338, positions 845 to 1338, positions 846 to 1338, positions 847 to 1338, positions 848 to 1338, positions 849 to 1338, positions 850 to 1338, positions 851 to 1338, positions 852 to 1338, positions 853 to 1338, positions 854 to 1338, positions 855 to 1338, positions 856 to 1338, positions 857 to 1338, positions 858 to 1338, positions 859 to 1338, positions 860 to 1338, positions 861 to 1338, positions 862 to 1338, positions 863 to 1338, positions 864 to 1338, positions 865 to 1338, positions 866 to 1338, positions 867 to 1338, positions 868 to 1338, positions 869 to 1338, positions 870 to 1338, positions 871 to 1338, positions 872 to 1338, positions 873 to 1338, positions 874 to 1338, positions 875 to 1338, positions 876 to 1338, positions 877 to 1338, positions 878 to 1338, positions 879 to 1338, positions 880 to 1338, positions 881 to 1338, positions 882 to 1338, positions 883 to 1338, positions 884 to 1338, positions 885 to 1338, positions 886 to 1338, positions 887 to 1338, positions 888 to 1338, positions 889 to 1338, positions 890 to 1338, positions 891 to 1338, positions 892 to 1338, positions 893 to 1338, positions 894 to 1338, positions 895 to 1338, positions 896 to 1338, positions 897 to 1338, positions 898 to 1338, positions 899 to 1338, positions 900 to 1338, positions 901 to 1338, positions 902 to 1338, positions 903 to 1338, positions 904 to 1338, positions 905 to 1338, positions 906 to 1338, positions 907 to 1338, positions 908 to 1338, positions 909 to 1338, positions 910 to 1338, positions 911 to 1338, positions 912 to 1338, positions 913 to 1338 or positions 914 to 1338 of GenBank Accession Number: NP 002010.2.

In that case, the Flt-1 decoy may include positions 718 to 782, positions 718 to 783, positions 718 to 784, positions 718 to 785, positions 718 to 786, positions 718 to 787, positions 718 to 788, positions 718 to 789, positions 718 to 790, positions 718 to 791, positions 718 to 792, positions 718 to 793, positions 718 to 794, positions 718 to 795, positions 718 to 796, positions 718 to 797, positions 718 to 798, positions 718 to 799, positions 718 to 800, positions 718 to 801, positions 718 to 802, positions 718 to 803, positions 718 to 804, positions 718 to 805, positions 718 to 806, positions 718 to 807, positions 718 to 808, positions 718 to 809, positions 718 to 810, positions 718 to 811, positions 718 to 812, positions 718 to 813, positions 718 to 814, positions 718 to 815, positions 718 to 816, positions 718 to 817, positions 718 to 818, positions 718 to 819, positions 718 to 820, positions 718 to 821, positions 718 to 822, positions 718 to 823, positions 718 to 824, positions 718 to 825, positions 718 to 826, positions 718 to 827, positions 718 to 828, positions 718 to 829, positions 718 to 830, positions 718 to 831, positions 718 to 832, positions 718 to 833, positions 718 to 834, positions 718 to 835, positions 718 to 836, positions 718 to 837, positions 718 to 838, positions 718 to 839, positions 718 to 840, positions 718 to 841, positions 718 to 842, positions 718 to 843, positions 718 to 844, positions 718 to 845, positions 718 to 846, positions 718 to 847, positions 718 to 848, positions 718 to 849, positions 718 to 850, positions 718 to 851, positions 718 to 852, positions 718 to 853, positions 718 to 854, positions 718 to 855, positions 718 to 856, positions 718 to 857, positions 718 to 858, positions 718 to 859, positions 718 to 860, positions 718 to 861, positions 718 to 862, positions 718 to 863, positions 718 to 864, positions 718 to 865, positions 718 to 866, positions 718 to 867, positions 718 to 868, positions 718 to 869, positions 718 to 870, positions 718 to 871, positions 718 to 872, positions 718 to 873, positions 718 to 874, positions 718 to 875, positions 718 to 876, positions 718 to 877, positions 718 to 878, positions 718 to 879, positions 718 to 880, positions 718 to 881, positions 718 to 882, positions 718 to 883, positions 718 to 884, positions 718 to 885, positions 718 to 886, positions 718 to 887, positions 718 to 888, positions 718 to 889, positions 718 to 890, positions 718 to 891, positions 718 to 892, positions 718 to 893, positions 718 to 894, positions 718 to 895, positions 718 to 896, positions 718 to 897, positions 718 to 898, positions 718 to 899, positions 718 to 900, positions 718 to 901, positions 718 to 902, positions 718 to 903, positions 718 to 904, positions 718 to 905, positions 718 to 906, positions 718 to 907, positions 718 to 908, positions 718 to 909, positions 718 to 910, positions 718 to 911, positions 718 to 912 or positions 718 to 913 of GenBank Accession Number: NP 002010.2.

In a specific embodiment, the Flt-1 decoy lacks all of the sequence corresponding to residues 781 to 1338 of GenBank Accession Number: NP_002010.2.

### Production of Flt-1 Decoy

A Flt-1 decoy as described in this document may be made by various means, for example by providing a full length Flt-1 sequence and removing the intracellular domain by recombinant means.

The Flt-1 decoy may therefore comprise a truncated Flt-1 polypeptide sequence which comprises the extracellular and transmembrane domains, but which lacks the intracellular signalling domain.

The skilled reader will appreciate that this is not the only way of providing a Flt-1 decoy without an intracellular domain and that other means of production are available. For example, an Flt-1 decoy may be assembled by providing a nucleic acid sequence encoding the extracellular domain of Flt-1 and providing a nucleic acid sequence encoding a transmembrane domain, for example, a transmembrane domain of Flt-1 in the context of an expression vector.

Expression from the expression vector will result in the production of an Flt-1 decoy in the form of a fusion protein comprising the extracellular domain of Flt-1 and a transmembrane domain. Such a fusion protein will of course lack an intracellular domain of Flt-1 and may function as a Flt-1 decoy.

Any suitable expression vector may be used. The expression vector may suitably be chosen so that it is capable of delivering the Flt-1 decoy to a place where it is needed, for example into a target cell, tissue, organ or organism.

For example, any of the viral vectors known in the art, for example, adenoviral expression vectors or adeno-associated expression vectors, may be employed for this purpose. Other suitable vectors include a plasmid construct naked or complexed with liposomes or polymersomes, a dumbbell DNA construct, or any other DNA-based construct described in the art.

The expression vector may comprise a sequence that inhibits the expression of VEGF, such as a VEGF-specific mirtron. This is described in detail below.

Flt-1 Decoy

A specific example of a Flt-1 decoy may be expressed from the nucleotide sequence:

(SEQ ID NO: 6)
ATGGTTTCTTACTGGGACACGGGGGTACTCCTTTGCGCACTTCTGTCTT
GTTTGCTTCTGACGGGGTCCTCTTCTGGGTCTAAGCTGAAAGATCCGGA
GCTGTCTCTGAAGGGCACGCAACACATCATGCAAGCTGGTCAAACTTTG
CATCTCCAGTGCCGGGGGGAGGCTGCGCATAAGTGGTCTTTGCCTGAAA
TGGTATCAAAGGAGTCCGAACGCCTCAGTATAACTAAAAGCGCATGTGG
TCGGAACGGAAAACAATTTTGTAGCACGCTCACACTCAATACTGCTCAA
GCCAATCATACCGGATTTTACTCTTGCAAATATCTTGCGGTACCGACCT
CTAAAAAGAAAGAAACCGAAAGCGCCATCTACATCTTCATTTCTGACAC
AGGCCGGCCATTTGTTGAAATGTATTCAGAGATCCCTGAAATCATCCAC
ATGACTGAGGGGCGAGAACTTGTTATACCCTGCCGGGTCACCAGTCCCA
ACATTACGGTGACCCTCAAAAAATTCCCACTGGATACGCTTATCCCGGA
CGGGAAGCGCATTATATGGGACTCTCGAAAAGGGTTTATCATTTCAAAT
GCCACGTACAAAGAAATCGGTCTGCTGACCTGCGAGGCCACGGTGAATG
GCCACTTGTATAAGACTAATTACCTCACTCACCGCCAGACAAACACAAT
TATCGATGTACAGATCTCCACACCTCGACCCGTTAAGCTGCTCAGAGGG
CATACACTTGTACTTAACTGCACAGCCACCACCCCGCTGAATACGAGAG
TACAGATGACCTGGTCATATCCGGACGAGAAGAACAAAAGAGCTTCCGT
GCGAAGGCGAATCGACCAGTCAAACTCCCATGCCAACATTTTCTACTCT
GTTCTGACGATCGACAAAATGCAGAACAAAGATAAGGGTTTGTACACTT
GTCGAGTCCGGAGTGGTCCATCTTTCAAAAGTGTAAATACTTCAGTGCA
TATCTACGATAAAGCCTTTATTACGGTCAAGCATCGAAAGCAACAAGTA
CTCGAAACTGTAGCAGGGAAACGCTCCTACCGGTTGTCTATGAAGGTAA
AGGCTTTTCCCAGCCCCGAAGTAGTGTGGCTCAAAGATGGGCTTCCGGC
GACGGAGAAGAGCGCTAGGTACTTGACAAGGGGCTACTCACTCATAATA
AAGGACGTGACGGAAGAGGACGCGGGGAATTATACAATACTTTTGTCCA
TAAAACAATCTAACGTTTTCAAAAACCTCACGGCGACTTTGATTGTCAA
TGTGAAACCTCAAATCTACGAGAAAGCTGTCTCTTCCTTCCCGGACCCA
GCGTTGTATCCACTCGGATCTAGGCAGATTCTCACCTGTACAGCCTACG
GGATACCGCAGCCTACTATTAAATGGTTTTGGCACCCATGTAACCATAA
CCACTCAGAAGCTCGCTGCGATTTCTGCTCTAATAATGAAGAGAGTTTC
ATACTTGACGCGGATTCCAACATGGGAAACCGCATTGAGTCAATTACCC
AACGGATGGCAATCATCGAAGGGAAGAACAAGATGGCCTCAACTCTCGT
GGTAGCAGATAGCCGAATTTCAGGAATATACATTTGTATCGCGTCTAAT
AAGGTAGGAACTGTCGGCCGAAATATATCCTTTTACATCACGGATGTCC
CCAACGGATTTCATGTAAATCTGGAAAAGATGCCCACAGAAGGAGAGGA
TCTGAAACTTTCCTGTACGGTAAATAAGTTCCTCTATCGCGACGTAACA
TGGATTTTGCTCCGGACCGTTAACAACCGCACCATGCATTACAGTATAT
CTAAGCAGAAGATGGCCATTACTAAAGAGCATTCTATTACACTGAACCT
CACTATCATGAATGTATCTCTTCAGGATAGTGGCACGTACGCGTGTCGC
GCTAGGAATGTGTATACTGGCGAGGAAATACTCCAGAAGAAAGAGATTA
CGATCAGGGACCAGGAGGCACCATACCTCCTGAGAAACCTTTCTGACCA
CACGGTGGCCATAAGTAGTAGTACGACACTTGATTGCCATGCGAACGGT
GTTCCGGAACCACAGATCACATGGTTTAAGAACAATCACAAAATCCAGC
AGGAGCCCGGGATCATACTTGGACCTGGGAGCTCCACGTTGTTTATTGA
AAGGGTTACCGAAGAGGACGAAGGGGTCTATCATTGTAAGGCAACAAAT
CAAAAGGGATCAGTTGAAAGTAGTGCATACTTGACCGTGCAAGGAACTA
GTGATAAATCCAACCTTGAGCTGATTACGTTGACGTGCACGTGCGTAGC
AGCTACCTTGTTCTGGCTGCTCTTGACCCTGTTCATTCGGAAAATGAAA
AGGTCCTCTAGTGAGATAAAAACTGACTACCTTTCCATAATAATGGACC
CGGACGAAGTTCCACTGGACGAACAGTGTGAACGCCTGCCGTACGACGC
GTCC

### Combination of Flt-1 and Mirtron

We specifically disclose a combination of a Flt-1 decoy as described in this document, together with a means to inhibit the expression or activity of VEGF. Such a VEGF inhibitory means may in particular comprise a RNA inhibitor of VEGF.

We specifically disclose a combination of a Flt-1 decoy and a mirtron capable of inhibiting VEGF.

Mirtrons are introns that form pre-microRNA hairpins after splicing, producing RNA interference (RNAi) effectors not processed by Drosha.

The Flt-1 decoy/VEGF mirtron combination may be provided as a simple combination of a polypeptide comprising a Flt-1 decoy and a nucleic acid encoding the VEGF mirtron.

The combination may also be provided as an expression vector comprising a nucleic acid encoding a mirtron having activity against VEGF and nucleic acid encoding a Flt-1 decoy.

In certain embodiments, the mirtron having activity against VEGF may be co-delivered with a transgene, i.e., a Flt-1 decoy, in a gene therapy package. The expression of the construct may be driven by tissue-specific promoters, which are known in the art.

The expression vector may comprise its elements in either order. The mirtron may for example be included in an intron in the Flt-1 encoding portion of the expression vector.

The mirtron capable of inhibiting VEGF may comprise any suitable sequence capable of inhibiting VEGF, for example, the expression or any activity of VEGF.

Methods of designing mirtron sequences for targeting specific polypeptide expression or activities are known in the art. A design algorithm for artificial mirtrons is described for example in Seow et al (2012). Artificial mirtron-mediated gene knockdown: Functional DMPK silencing in mammalian cells. RNA. 2012 July; 18(7): 1328-1337.

Specific anti-VEGF mirtron sequences which may be used in the combinations described here are set out for example in Kock et al. (2015). Functional VEGFA knockdown with artificial 3′-tailed mirtrons defined by 5′ splice site and branch point. Nucleic Acids Research, Volume 43, Issue 13, 27 Jul. 2015, Pages 6568-6578.

For example, the mirtron capable of inhibiting VEGF may comprise the sequence

(SEQ ID NO: 1)
GTAAATGTATGTATGTGGGTGTTCAAGAGACACCCACACACATACATCT
CAGTTTTTTCTCTTTCTTTCAG.

Alternatively, or in addition, the mirtron capable of inhibiting VEGF may comprise the sequence

(SEQ ID NO: 2)
GTAGATTATGCGGATTAAATTTCAAGAGAGTTTGATCCGCATAATCTGT
CAGTTTTTTCTCTTTCTTTCAG.

### Diseases and Conditions

Any disease associated with VEGF expression, for example, elevated VEGF expression, may be targeted with the Flt-1 decoy, optionally in combination with a mirtron capable of inhibiting VEGF.

Such targeting may comprise treatment, alleviation, prophylaxis, prevention or reduction in symptoms of the specific disease or condition.

Diseases associated with VEGF expression are known in the art and are described for example in Shibuya (2014). VEGF-VEGFR Signals in Health and Disease. Biomol Ther (Seoul). 2014 January; 22(1): 1-9.

Suitable targets may include tumour cells and other proliferative cells.

The term proliferative disorder is used herein in a broad sense to include any disorder that requires control of the cell cycle. In particular, a proliferative disorder includes malignant and pre-neoplastic disorders. The methods and compositions described here are especially useful in relation to treatment or diagnosis of adenocarcinomas such as: small cell lung cancer, and cancer of the kidney, uterus, prostrate, bladder, ovary, colon and breast. For example, malignancies which may be treatable include acute and chronic leukemias, lymphomas, myelomas, sarcomas such as Fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, colon carcinoma, ovarian cancer, glioma, prostate cancer, pancreatic cancer, breast cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

Treatable diseases include in particular liver, breast, sarcoma, lung, prostate, bladder, kidney, melanoma, pancreatic, endometrial, colorectal and thyroid cancer.

A Flt-1 decoy, optionally in combination with a mirtron capable of inhibiting VEGF, as described here may be used to treat any disease of aberrant neovascularisation.

Further diseases which may be targeted include macular degeneration, corneal neovascularization, diabetic retinopathy, retinal vein occlusions, and retinopathy of prematurity (Campochiaro P A (2013). Ocular neovascularization. J Mol Med (Berl). 2013 March; 91(3):311-21). Macular degeneration, also known as age-related macular degeneration (AMD or ARMD), is a medical condition which may result in blurred or no vision in the center of the visual field.

Neovascular or exudative AMD, the “wet” form of advanced AMD, causes vision loss due to abnormal blood vessel growth (choroidal neovascularization) in the choriocapillaris, through Bruch's membrane.

The proliferation of abnormal blood vessels in the retina is stimulated by vascular endothelial growth factor (VEGF). Accordingly, wet AMD may be targeted by a Flt-1 decoy, optionally in combination with a mirtron capable of inhibiting VEGF, as described here.

### FLT-1

The following text is adapted from the OMIM entry for Flt-1 (https://www.omim org/entry/165070?search=flt-1), contributors Ada Hamosh, George E. Tiller, Ada Hamosh, Ada Hamosh, Patricia A. Hartz, John A. Phillips, III, Paul J. Converse, Ada Hamosh, Ada Hamosh, Marla J. F. O'Neill, Ada Hamosh, Paul J. Converse, Patricia A. Hartz, Marla J. F. O'Neill, Ada Hamosh, Ada Hamosh, John A. Phillips, III, John A. Phillips, III, Stylianos E. Antonarakis and Jennifer P. Macke.

Flt-1 is also known as FMS-Related Tyrosine Kinase 1, FLT1, Vascular Endothelial Growth Factor/Vascular Permeability Factor Receptor, Vascular Endothelial Growth Factor Receptor 1 and VEGFR1. It has gene symbol FLT1, cytogenetic location 13q12.3 and genomic coordinates (GRCh38): 13:28,300,345-28,495,127

Oncogene FLT belongs to the src gene family and is related to oncogene ROS. Like other members of this family, it shows tyrosine protein kinase activity that is important for the control of cell proliferation and differentiation. The sequence structure of the FLT gene resembles that of the FMS gene (164770); hence, Yoshida et al. (1987) proposed the name FLT as an acronym for FMS-like tyrosine kinase.

Shibuya et al. (1990) cloned full-length FLT1 from normal human placenta RNA. The deduced 1,338-amino acid protein has a calculated molecular mass of 150.6 kD. It has a 758-amino acid extracellular domain, followed by a 22-amino acid transmembrane region and a 558-amino acid cytoplasmic region containing a cluster of basic amino acids and a tyrosine kinase domain. The kinase domain has 3 conserved glycines (G834, G836, and G839), a conserved lysine (K861) involved in ATP binding, and a putative tyrosine autophosphorylation site (Y1053). Northern blot analysis detected a transcript of about 8.0 kb that was highly expressed in placenta and more weakly expressed in liver, muscle, and kidney. Weak expression was also detected in kidney cell lines and a choriocarcinoma cell line. Rat Flt1 was widely expressed in normal tissues, with highest expression in lung.

Kendall and Thomas (1993) stated that the extracellular ligand-binding region of full-length FLT1 has an N-terminal signal peptide followed by 7 immunoglobulin-like domains. Using a probe encoding the extracellular domain of FLT1 to screen a human umbilical vein endothelial cell (HUVEC) cDNA library, Kendall and Thomas (1993) obtained 2 cDNAs with different 5-prime and 3-prime ends. Both cDNAs encode the same truncated soluble form of FLT1, which Kendall and Thomas (1993) designated sFLT. The deduced 687-amino acid protein contains the secretory leader sequence and 6 of the 7 N-terminal extracellular immunoglobulin domains of full-length FLT1, but it lacks the transmembrane and kinase domains and terminates in 31 unique C-terminal amino acids. It also has 12 N-glycosylation sites. Sequencing of mature sFLT revealed that the N terminus began with ser27, suggesting cleavage of the leader sequence.

By EST database analysis, followed by RACE of preeclamptic (see 189800) placenta, Sela et al. (2008) cloned an additional splice variant encoding a soluble form of FLT1 that they called sFLT1-14. This transcript has a unique 3-prime end due to use of a splice acceptor site within intron 14. The deduced 733-amino acid protein has only the extracellular domain of full-length FLT1, and it has a unique C terminus relative to the sFLT1 isoform. Northern blot analysis showed that endothelial cells from human saphenous vein and radial artery expressed full-length FLT1 and sFLT1, whereas vascular smooth muscle cells from the same vessels expressed only sFLT1-14. Western blot analysis of transfected HeLa cells detected a cell-associated sFLT1-14 protein at an apparent molecular mass of 115 kD and a secreted form at 130 kD.

Gene Function

Kendall and Thomas (1993) showed that recombinant sFLT bound vascular endothelial growth factor (VEGF; 192240) with high affinity. In culture, sFLT competed with FLT1 receptors on HUVECs for binding to radiolabelled VEGF. Furthermore, sFLT eliminated VEGF-induced mitogenesis of HUVECs in a concentration-dependent manner.

Kendall et al. (1996) showed that endogenous sFLT identified in the conditioned culture medium of HUVECs bound VEGF with high affinity comparable to that of recombinant sFLT. They found that sFLT1 and FLK1 (KDR; 191306) formed heterodimers in vitro. Because sFLT1 had a higher affinity for VEGF than for FLK1, Kendall et al. (1996) suggested that sFLT1 may function as an inhibitor of VEGF response.

Wiesmann et al. (1997) reported the results of domain deletion studies of the extracellular portion of FLT1. They showed that FLT1 domains 2 and 3 are necessary and sufficient for binding VEGF with near-native affinity and that domain 2 alone binds only 60-fold less tightly than wildtype.

He et al. (1999) described a mouse cDNA sequence encoding sFLT1 that is a potent antagonist to VEGF and showed its in vivo production. In situ hybridization and Northern blot analysis with probes specific for sFLT1 or FLT1 showed that the relative abundance of their mRNAs changed markedly in spongiotrophoblast cells in the placenta as gestation progressed. On day 11 of pregnancy, sFLT1 mRNA was undetectable but FLT1 was readily apparent, and by day 17 sFLT1 mRNA was abundant but FLT1 was barely detectable. The authors concluded that these data suggest a novel mechanism of regulation of angiogenesis by alternative splicing of FLT1 pre-mRNA.

Wulff et al. (2001) examined the effects of a soluble truncated form of FLT1, vascular endothelial growth factor trapA40 (VEGF trap), in a primate model to determine its ability to prevent the onset of luteal angiogenesis or intervene with the ongoing process. Marmosets were treated from the day of ovulation until luteal day 3 (prevention regimen) or on luteal day 3 for 1 day (intervention regimen). After both treatments, intense luteal endothelial proliferation was suppressed, a concomitant decrease in endothelial cell area confirmed the inhibition of vascular development, and a marked fall in plasma progesterone levels showed that luteal function was compromised. The authors concluded that the VEGF trap can prevent luteal angiogenesis and inhibit the established process with resultant suppression of luteal function; that luteal FLT mRNA expression is dependent upon VEGF; and that VEGF inhibition results in abortive increases in expression of VEGF, angiopoietin-2 (601922), and TIE2 (600221).

To explore the role of sinusoidal endothelial cells in the adult liver, LeCouter et al. (2003) studied the effects of VEGF receptor activation on mouse hepatocyte growth. Delivery of VEGFA increased liver mass in mice but did not stimulate growth of hepatocytes in vitro unless liver sinusoidal endothelial cells were also present in the culture. Hepatocyte growth factor (HGF; 142409) was identified as one of the liver sinusoidal endothelial cell-derived paracrine mediators promoting hepatocyte growth. Selective activation of VEGFR1 stimulated hepatocyte but not endothelial proliferation in vivo and reduced liver damage in mice exposed to a hepatotoxin.

Autiero et al. (2003) reported that placental growth factor (PGF; 601121) regulates inter- and intramolecular cross-talk between the VEGF receptor tyrosine kinases FLT1 and FLK1. Activation of FLT1 by PGF resulted in intermolecular transphosphorylation of FLK1, thereby amplifying VEGF-driven angiogenesis through FLK1. Even though VEGF and PGF both bind FLT1, PGF uniquely stimulated the phosphorylation of specific FLT1 tyrosine residues and the expression of distinct downstream target genes. Furthermore, the VEGF/PGF heterodimer activated intramolecular VEGF receptor cross-talk through formation of FLK1/FLT1 heterodimers. Autiero et al. (2003) concluded that the inter- and intramolecular VEGF receptor cross-talk is likely to have therapeutic implications, as treatment with VEGF/PGF heterodimer or a combination of VEGF plus PGF increased ischemic myocardial angiogenesis in a mouse model that was refractory to VEGF alone.

In preeclamptic (see 189800) women, Maynard et al. (2003) found increased sFLT1 associated with decreased circulating levels of free VEGF and PGF, resulting in endothelial dysfunction in vitro that was rescued by exogenous VEGF and PGF. Administration of sFLT1 to pregnant rats induced hypertension, proteinuria, and glomerular endotheliosis, the classic lesion of preeclampsia. Maynard et al. (2003) suggested that excess circulating sFLT1 contributes to the pathogenesis of preeclampsia.

In 120 preeclamptic women and 120 matched, normotensive controls, Levine et al. (2004) measured serum levels of the angiogenic factors sFLT1, PGF, and VEGF throughout pregnancy. During the last 2 months of pregnancy in the normotensive controls, the level of sFLT1 increased and the level of PGF decreased; these changes occurred earlier and were more pronounced in the women in whom preeclampsia later developed. At the onset of clinical disease, the mean serum level of sFLT1 in the preeclamptic women was significantly higher than that of controls with foetuses of similar gestational age (p less than 0.001). PGF levels were significantly lower in women who later had preeclampsia than in controls, beginning at 13 to 16 weeks of gestation (p=0.01), with the greatest difference occurring during the weeks before the onset of preeclampsia, coincident with an increase in the sFLT1 level. Levine et al. (2004) concluded that increased levels of sFLT1 and reduced levels of PGF predict the subsequent development of preeclampsia.

Kaplan et al. (2005) demonstrated that bone marrow-derived hematopoietic progenitor cells that express VEGFR1 home to tumor-specific premetastatic sites and form cellular clusters before the arrival of tumor cells. Preventing VEGFR1 function using antibodies or by the removal of VEGFR1-positive cells from the bone marrow of wildtype mice abrogated the formation of these premetastatic clusters and prevented tumor metastasis, whereas reconstitution with selected Id3-competent VEGFR1-positive cells established cluster formation and tumor metastasis in Id3 knockout mice. Kaplan et al. (2005) also showed that VEGFR1-positive cells express VLA4, also known as integrin alpha-4-beta-1 (see 192975), and that tumor-specific growth factors upregulate fibronectin, a VLA4 ligand, in resident fibroblasts, providing a permissive niche for incoming tumor cells. Conditioned media obtained from distinct tumor types with unique patterns of metastatic spread redirected fibronectin expression and cluster formation, thereby transforming the metastatic profile. Kaplan et al. (2005) concluded that their findings demonstrated a requirement for VEGFR1-positive hematopoietic progenitors in the regulation of metastasis, and suggested that expression patterns of fibronectin and VEGFR1-positive-VLA4-positive clusters dictate organ-specific tumor spread.

Ambati et al. (2006) showed that the cornea expresses soluble VEGF receptor-1, also known as SFLT1, and that suppression of this endogenous VEGF-A trap by neutralizing antibodies, RNA interference, or Cre-lox-mediated gene disruption abolishes corneal avascularity in mice. The spontaneously vascularized corneas of corn1 (see 609114) and Pax6 heterozygous mice (see 607108) and Pax6 heterozygous patients with aniridia are deficient in SFLT1, and recombinant Sflt1 administration restores corneal avascularity in corn1 and Pax6 heterozygous mice. Manatees, the only known creatures uniformly to have vascularized corneas, do not express sflt1, whereas the avascular corneas of dugongs (also members of the order Sirenia), elephants, the closest extant terrestrial phylogenetic relatives of manatees, and other marine mammals (dolphins and whales) contain sflt1, indicating that it has a crucial, evolutionarily conserved role.

Clinical trials of small interfering RNA (siRNA) targeting vascular endothelial growth factor A (VEGFA; 192240) or its receptor VEGFR1 in patients with blinding choroidal neovascularization (CNV) from age-related macular degeneration are premised on gene silencing by means of intracellular RNA interference (RNIi). Kleinman et al. (2008) showed instead that CNV inhibition is a siRNA-class effect: 21-nucleotide or longer siRNAs targeting nonmammalian genes, non-expressed genes, nongenomic sequences, pro- and antiangiogenic genes, and RNAi-incompetent siRNAs all suppressed CNV in mice comparably to siRNA targeting Vegfa or Vegfr1 without off-target RNAi or interferon-alpha/beta activation. Nontargeted (against nonmammalian genes) and targeted (against Vegfa or Vegfr1) siRNA suppressed CNV via cell surface toll-like receptor-3 (TLR3; 603029), its adaptor TRIF, and induction of interferon-gamma and interleukin-12 (see 161560). Nontargeted siRNA suppressed dermal neovascularization in mice as effectively as Vegfa siRNA. siRNA-induced inhibition of neovascularization required a minimum length of 21 nucleotides, a bridging necessity in a modelled 2:1 TLR3-RNA complex. Choroidal endothelial cells from people expressing the TLR3 coding variant 412FF were refractory to extracellular siRNA-induced cytotoxicity, facilitating individualized pharmacogenetic therapy. Multiple human endothelial cell types expressed surface TLR3, indicating that generic siRNAs might treat angiogenic disorders that affect 8% of the world's population, and that siRNAs might induce unanticipated vascular or immune effects.

Using porcine aortic endothelial cells expressing human VEGFR2 (KDR), Sela et al. (2008) showed that both sFLT1 and sFLT1-14 inhibited VEGFR2 phosphorylation in a dose-dependent manner Northern blot analysis of normal placenta showed that expression of soluble FLT1 transcripts increased progressively, and the increase was predominantly due to increased expression of sFLT1-14, which became the dominant transcript at the beginning of the second trimester. Expression of sFLT1-14 was further elevated in preeclamptic placentas. In situ hybridization showed that sFLT1-14 was expressed in syncytial knots characteristic of preeclamptic placentas, as well as in the many fewer syncytial knots formed in older normal placentas. Expression of sFLT1-14 was much lower in normal syncytiotrophoblasts. ELISA analysis detected sFLT1-14 in sera of preeclamptic women.

Stefater et al. (2011) showed that during development, retinal myeloid cells produce Wnt ligands to regulate blood vessel branching. In the mouse retina, where angiogenesis occurs postnatally, somatic deletion in retinal myeloid cells of the Wnt ligand transporter Wntless results in increased angiogenesis in the deeper layers. Stefater et al. (2011) also showed that mutation of Wnt5a and Wnt11 results in increased angiogenesis and that these ligands elicit retinal myeloid cell responses via a noncanonical Wnt pathway. Using cultured myeloid-like cells and retinal myeloid cell somatic deletion of Flt1, Stefater et al. (2011) showed that Flt1, a naturally occurring inhibitor of VEGF, is an effector of Wnt-dependent suppression of angiogenesis by retinal myeloid cells. Stefater et al. (2011) concluded that resident myeloid cells can use a noncanonical, Wnt-Flt1 pathway to suppress angiogenic branching.

Zhang et al. (2018) showed that preventing lacteal chylomicron uptake by inducible endothelial genetic deletion of neuropilin-1 (NRP1; 602069) and Vegfr1 (Flt1) renders mice resistant to diet-induced obesity. Absence of Nrp1 and Flt1 receptors increased Vegfa bioavailability and signalling through Vegfr2, inducing lacteal junction zippering and chylomicron malabsorption. Restoring permeable lacteal junctions by Vegfr2 and vascular endothelial cadherin (VE-cadherin; 601120) signalling inhibition rescued chylomicron transport in mutant mice. Zippering of lacteal junctions by disassembly of cytoskeletal VE-cadherin anchors prevented chylomicron uptake in wildtype mice.

Gene Structure

Sela et al. (2008) reported that the FLT1 gene contains at least 30 coding exons, all of which are included in the full-length FLT1 transcript. Use of alternative splice acceptor sites within introns 13 and 14 produces transcripts encoding the truncated soluble isoforms sFLT1 and sFLT1-14, respectively.

Mapping

Satoh et al. (1987) and Yoshida et al. (1987) mapped FLT to 13q12 by probing of DNA from a panel of human-mouse somatic cell hybrids and by in situ hybridization. By the isolation and analysis of a YAC containing the FLT1 and FLT3 genes, Imbert et al. (1994) confirmed their close physical linkage. FLT1 and FLT3 are linked in a head-to-tail configuration and are separated by about 150 kb. Imbert et al. (1994) found that the region contains 3 CpG islands, 2 of which were thought to correspond to FLT1 and FLT3 and the third to a putative, unidentified receptor-type tyrosine kinase (RTK) gene. They referred to studies performed by fluorescence in situ hybridization using the YAC as a probe.

Biochemical Features

Crystal Structure

Wiesmann et al. (1997) reported the crystal structure to 1.7-angstrom resolution of the complex between the receptor-binding domain of VEGF and FLT1 domain 2. The crystal structure of the complex between VEGF and the second domain of FLT1 shows domain 2 in a predominantly hydrophobic interaction with the ‘poles’ of the VEGF dimer. Based on this structure and on mutational data, Wiesmann et al. (1997) presented a model of VEGF bound to the first 4 domains of FLT1.

Role in Peripartum Cardiomyopathy Susceptibility

To test the idea that angiogenic signalling can cause cardiac dysfunction in pregnant women, Patten et al. (2012) studied women with preeclampsia (see 189800) who had compromised VEGF signalling due to high serum levels of antiangiogenic sFLT1. Cardiac function was evaluated noninvasively by measuring the myocardial performance index (MPI) and other indices of cardiac function with cardiac echocardiography. Women with preeclampsia had markedly increased serum levels of sFLT1 (p=0.005), as previously shown by Levine et al. (2004). Notably, women with preeclampsia also had a markedly abnormal MPI and other indicators of cardiac diastolic dysfunction. The MPI correlated with levels of circulating sFLT1 (R=0.59, p=0.003). To test this idea directly, Patten et al. (2012) delivered sFLT1 systemically to pregnant mice by intravenous injection of adenoviruses expressing sFLT1 and examined MPI using high-resolution murine echocardiography. sFLT1 caused significant increases in MPI in these mice within 10 days. Patten et al. (2012) concluded that their data, taken together with published observations in patients receiving antiangiogenic therapies, strongly suggested that elevated sFLT1 causes cardiac dysfunction in women with preeclampsia.

Patten et al. (2012) collected plasma from women with peripartum cardiomyopathy (PPCM; 614670) 4 to 6 weeks postpartum and measured sFLT1 levels. sFLT1 levels usually return to normal within 48 to 72 hours after delivery. sFLT1 levels were elevated in a large subset of these PPCM patients (p=0.002), remaining up to 5- to 10-fold higher than the levels of control participants. Postpartum sFLT1 levels can remain slightly higher in subjects with preeclampsia, but the levels found by Patten et al. (2012) in this subset of PPCM patients were notably higher. Thus, the findings were consistent with the idea that a substantial percentage of PPCM subjects have been exposed to preeclampsia, and that secretion of sFLT1 persists inappropriately postpartum. Among Harvard teaching hospitals included in the study by Patten et al. (2012), 33% of the last 75 cases of PPCM were associated with preeclampsia, markedly more than the population rate of 3 to 5%. The persisting extraplacental source of sFLT1 in the postpartum period was not known, and Patten et al. (2012) suggested that this source may include placental remnants, circulating mononuclear cells, or shed syncytial microparticles.

Patten et al. (2012) suggested that PPCM is caused by a 2-hit combination of, firstly, systemic antiangiogenic signals during late pregnancy, and, secondly, a host susceptibility marked by insufficient local proangiogenic defences in the heart. The first hit explains why PPCM is a disease of the late gestational period, which is precisely when circulating antiangiogenic factors such as sFLT1 peak in pregnancy. The first hit is also worse in preeclampsia, which is characterized by markedly elevated sFLT1 levels. Patten et al. (2012) hypothesized a number of possible second hits, including abnormal PGC1-alpha, myocarditis, immune activation, viral infection, and/or autoantibodies. Patten et al. (2012) concluded that their data supported the idea that PPCM is partly a 2-hit vascular disease due to imbalances in angiogenic signalling, and that antiangiogenic states such as preeclampsia or multiple gestation substantially worsen the severity of the disease. Their observations also suggested that proangiogenic therapies such as exogenous VEGF121, or removal of sFLT1 itself, may therefore be beneficial in PPCM.

Molecular Genetics

Fetal Loss in Placental Malaria

Placental malaria (PM; see 611162) is caused by P. falciparum-infected erythrocytes adhering to chondroitin sulphate A and sequestering in the maternal circulation of the placenta. The highest rates of PM and foetal loss are in first-time mothers. Muehlenbachs et al. (2008) showed that a dinucleotide repeat polymorphism approximately 3 kb downstream of the last exon of FLT1, rs3138582, was expressed within the FLT1 UTR. They classified dinucleotide repeat polymorphisms with more than 27 repeats as the long (L) allele and those with 27 repeats or less as the short (S) allele and investigated the relationship of FLT1 repeat length to poor outcomes caused by PM in Tanzanian mother-infant pairs. The newborn genotype distribution differed by birth order, with newborns of first-time mothers having a genotype distribution that was out of Hardy-Weinberg equilibrium during peak PM season, when significantly fewer SS homozygotes were born to these mothers. First-time mothers who were SS homozygous were more likely to report prior foetal loss than those with other genotypes. First-time mothers of SS homozygous offspring had higher plasma FLT1 levels during PM compared with first-time mothers of SL or LL offspring. Peripheral blood mononuclear cells stimulated with lipopolysaccharide showed increased expression of FLT1mRNA and protein in infants with SS and SL genotypes than in those with LL genotype. Muehlenbachs et al. (2008) suggested that foetal genes such as FLT1 may modify maternal inflammation and may be under natural selection secondary to malaria.

Thyroid Cancer

Liu et al. (2008) explored a wide-range genetic basis for the involvement of genetic alterations in receptor tyrosine kinases (RTKs) and phosphatidylinositol 3-kinase (PI3K)/Akt and MAPK pathways in anaplastic thyroid cancer (ATC) and follicular thyroid cancer (FTC; see 188470). They found frequent copy gains of RTK genes including EGFR, and VEGFR1, and in PIK3CA and PIK3CB in the P13K/Akt pathway. Copy number gain of VEGFR1 was found in 20 of 44 ATCs (46%) and 26 of 59 FTCs (44%). RTK gene copy gains were preferentially associated with phosphorylation of Akt, suggesting their dominant role in activating the P13K/Akt pathway. Liu et al. (2008) concluded that genetic alterations in the RTKs and P13K/Akt and MAPK pathways are extremely prevalent in ATC and FTC, providing a strong genetic basis for an extensive role of these signalling pathways and the development of therapies targeting these pathways for ATC and FTC, particularly the former.

Animal Model

VEGF and its high-affinity binding receptors, the tyrosine kinases FLK1 and FLT1, are thought to be important for the development of embryonic vasculature. Studying transgenic mice in whom the Flk1 gene was disrupted, Shalaby et al. (1995) demonstrated a total failure of embryonic mice to develop blood vessels and failure of blood island formation in the yolk sac. Fong et al. (1995) reported that in mice Flt1 is essential for the organization of embryonic vasculature, but is not essential for endothelial cell differentiation. Transgenic mouse embryos homozygous for a targeted mutation in the Flt1 locus formed endothelial cells in both embryonic and extraembryonic regions, but assembled these cells into abnormal vascular channels and died in utero at mid-somite stages. At earlier stages, the blood islands of homozygous mice were abnormal, with angioblasts in the interior as well as on the periphery. Fong et al. (1995) suggested that the Flt1 signalling pathway may regulate normal endothelial cell-cell or cell-matrix interactions during vascular development.

Niida et al. (2005) stated that Csf1-null mice are osteopetrotic and that those null for the Flt1 gene show mild osteoclast reduction without bone marrow suppression. They created double-knockout mice that exhibited severe osteoclast deficiency and did not have sufficient osteoclasts to form the bone marrow cavity. The cavity of double-knockout mice was gradually replaced with fibrous tissue, resulting in severe marrow hypoplasia and extramedullary hematopoiesis. The number of osteoblasts was also decreased. Niida et al. (2005) concluded that FLT1 and CSF1 receptors play predominant roles in osteoclastogenesis and the maintenance of bone marrow function.

Using a transgenic mouse model of rheumatoid arthritis (RA) and antibodies to Vegf, Vegfr1, and Vegfr2, De Bandt et al. (2003) found that synovial cells from arthritic joints expressed all 3 proteins. Treatment with anti-Vegfr1 strongly attenuated the disease throughout the study period, whereas anti-Vegf transiently delayed disease onset and anti-Vegfr2 treatment had no effect. Histologic analysis showed that treatment with a VEGFR1 tyrosine kinase inhibitor nearly abolished the disease. De Bandt et al. (2003) concluded that VEGF is a key factor in pannus development, acting through the VEGFR1 pathway, and they proposed that VEGFR1 inhibitors may be useful in the treatment of RA.

In laser-injury studies in mice, Nozaki et al. (2006) observed that injury-induced choroidal neovascularization (CNV) was increased by excess Vegf before injury but was suppressed by Vegf after injury. This effect was mediated via Vegfr1 activation and Vegfr2 deactivation: excess Vegf increased CNV before injury because Vegfr1 activation was silenced by Sparc, and a transient decline in Sparc after injury created a temporal window in which Vegf signalling was routed primarily through Vegfr1.

Since Flt1 is a decoy receptor for vascular endothelial growth factor (VEGF), both homozygous (Flt1−/−) and heterozygous (Flt1+/−) Flt1 gene knockout mice display increased endothelial cell proliferation and vascular density during embryogenesis. In addition to its presence in muscle, dystrophin is also found in vasculature, and its absence results in vascular deficiency and abnormal blood flow. To create a mouse model of Duchenne muscular dystrophy (DMD) with increased vasculature, Verma et al. (2010) crossed mdx mice with Flt1knockout mice. Flt1+/− and mdx:Flt1+/− adult mice displayed a developmentally increased vascular density in skeletal muscle compared with wildtype and mdx mice, respectively. The mdx:Flt1+/− mice showed improved muscle histology compared with mdx mice, with decreased fibrosis, calcification, and membrane permeability. Functionally, the mdx:Flt1+/− mice had an increase in muscle blood flow and force production compared with mdx mice. Because utrophin is upregulated in mdx mice and can compensate for the lacking function of dystrophin, Verma et al. (2010) created a triple-mutant mouse (mdx:utrophin−/−:Flt1+/−). The mdx:utrophin−/−:Flt1+/− mice also displayed improved muscle histology and significantly higher survival rates compared with mdx:utrophin−/− mice, which showed more severe muscle phenotypes than mdx mice. Verma et al. (2010) suggested that increasing the vasculature in DMD may ameliorate the histologic and functional phenotypes associated with this disease.

### Polypeptides

The methods and compositions described here make use of polypeptides, which are described in detail below.

Such polypeptides may include Flt-1, a VEGF binding domain of Flt-1, a membrane anchoring domain, a transmembrane domain of Flt1, an intracellular domain, an intracellular signalling domain of Flt-1, etc.

As used here, the term “Flt-1 polypeptide” is intended to refer to a sequence having GenBank Accession number NP 002010.2.

A “Flt-1 polypeptide” may comprise or consist of a human Flt-1 polypeptide, such as the sequence having accession number NP_002010.2.

Homologues variants and derivatives thereof of any, some or all of these polypeptides are also included.

A “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-inking, cyclization, disulfide bond formation, demethylation, formation of covalent cross-inks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F, Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et aL, “Protein Synthesis: Posttranslational Modifications and Aging”, Ann NY Acad Sci (1992) 663:48-62.

The term “polypeptide” includes the various synthetic peptide variations known in the art, such as a retroinverso D peptides. The peptide may be an antigenic determinant and/or a T-cell epitope. The peptide may be immunogenic in vivo. The peptide may be capable of inducing neutralising antibodies in vivo.

The resultant amino acid sequence may have one or more activities, such as biological activities. In particular, the term “homologue” covers identity with respect to structure and/or function providing the resultant amino acid sequence has a relevant activity, such as Flt-1 activity. With respect to sequence identity (i.e. similarity), there may be at least 70%, such as at least 75%, such as at least 85%, such as at least 90% sequence identity. There may be at least 95%, such as at least 98%, sequence identity. These terms also encompass polypeptides derived from amino acids which are allelic variations of the nucleic acid sequence.

Where reference is made to the “activity” or “biological activity” of a polypeptide such as Flt-1, these terms are intended to refer to the metabolic or physiological function of Flt-1, including similar activities or improved activities or these activities with decreased undesirable side effects. Also included are antigenic and immunogenic activities of Flt-1. Examples of such activities, and methods of assaying and quantifying these activities, are known in the art, and are described in detail elsewhere in this document.

For example, such activities may include any one or more of the following: VEGF binding activity. Assays for these activities are known in the art.

### Other Polypeptides

Variants, homologues, derivatives and fragments of the polypeptides described here are also of use in the methods and compositions described here.

The terms “variant”, “homologue”, “derivative” or “fragment” in relation to a polypeptide include any substitution of, variation of, modification of, replacement of, deletion of or addition of one (or more) amino acid from or to a sequence. Unless the context admits otherwise, references to “Flt-1” includes references to such variants, homologues, derivatives and fragments of Flt-1.

As used herein a “deletion” is defined as a change in either nucleotide or amino acid sequence in which one or more nucleotides or amino acid residues, respectively, are absent. As used herein an “insertion” or “addition” is that change in a nucleotide or amino acid sequence which has resulted in the addition of one or more nucleotides or amino acid residues, respectively, as compared to the naturally occurring substance. As used herein “substitution” results from the replacement of one or more nucleotides or amino acids by different nucleotides or amino acids, respectively.

Polypeptides such as Flt-1 as described here may also have deletions, insertions or substitutions of amino acid residues which produce a silent change and result in a functionally equivalent amino acid sequence. Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the table below. Amino acids in the same block in the second column and in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Polypeptides may further comprise heterologous amino acid sequences, typically at the N-terminus or C-terminus, such as the N-terminus. Heterologous sequences may include sequences that affect intra or extracellular protein targeting (such as leader sequences). Heterologous sequences may also include sequences that increase the immunogenicity of the polypeptide and/or which facilitate identification, extraction and/or purification of the polypeptides. Another heterologous sequence that may be used is a polyamino acid sequence such as polyhistidine which may be N-terminal. A polyhistidine sequence of at least 10 amino acids, such as at least 17 amino acids but fewer than 50 amino acids may be employed.

The polypeptides may be in the form of the “mature” protein or may be a part of a larger protein such as a fusion protein. It is often advantageous to include an additional amino acid sequence which contains secretory or leader sequences, pro-sequences, sequences which aid in purification such as multiple histidine residues, or an additional sequence for stability during recombinant production.

Polypeptides as described here are advantageously made by recombinant means, using known techniques. However they may also be made by synthetic means using techniques well known to skilled persons such as solid phase synthesis. Such polypeptides may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences, such as a thrombin cleavage site. The fusion protein may be one which does not hinder the function of the protein of interest sequence.

The polypeptides may be in a substantially isolated form. This term is intended to refer to alteration by the hand of man from the natural state. If an “isolated” composition or substance occurs in nature, it has been changed or removed from its original environment, or both. For example, a polynucleotide, nucleic acid or a polypeptide naturally present in a living animal is not “isolated,” but the same polynucleotide, nucleic acid or polypeptide separated from the coexisting materials of its natural state is “isolated”, as the term is employed herein.

It will however be understood that the protein may be mixed with carriers or diluents which will not interfere with the intended purpose of the protein and still be regarded as substantially isolated. A polypeptide may also be in a substantially purified form, in which case it will generally comprise the protein in a preparation in which more than 90%, for example, 95%, 98% or 99% of the protein in the preparation is a polypeptide.

By aligning polypeptide sequences from different species, it is possible to determine which regions of the amino acid sequence are conserved between different species (“homologous regions”), and which regions vary between the different species (“heterologous regions”).

The polypeptides may therefore comprise a sequence which corresponds to at least part of a homologous region. A homologous region shows a high degree of homology between at least two species. For example, the homologous region may show at least 70%, at least 80%, at least 90% or at least 95% identity at the amino acid level using the tests described above. Peptides which comprise a sequence which corresponds to a homologous region may be used in therapeutic strategies as explained in further detail below. Alternatively, the peptide may comprise a sequence which corresponds to at least part of a heterologous region. A heterologous region shows a low degree of homology between at least two species.

### Homologues

The polypeptides disclosed for use include homologous sequences obtained from any source, for example related viral/bacterial proteins, cellular homologues and synthetic peptides, as well as variants or derivatives thereof. Thus polypeptides also include those encoding homologues of the polypeptide from other species including animals such as mammals (e.g. mice, rats or rabbits), especially primates, more especially humans. More specifically, homologues include human homologues.

In the context of this document, a homologous sequence is taken to include an amino acid sequence which is at least 15, 20, 25, 30, 40, 50, 60, 70, 80 or 90% identical, such as at least 95 or 98% identical at the amino acid level, for example over at least 50 or 100, 110, 115, 120, 125, 130, 135, 140, 141, 142, 143, 144, 145, 146, 147, 148 or 149 amino acids with the sequence of a relevant polypeptide sequence.

In particular, homology should typically be considered with respect to those regions of the sequence known to be essential for protein function rather than non-essential neighbouring sequences. This is especially important when considering homologous sequences from distantly related organisms.

Although homology can also be considered in terms of similarity (i e amino acid residues having similar chemical properties/functions), in the context of the present document homology may be expressed in terms of sequence identity.

Homology comparisons can be conducted by eye, or more usually, with the aid of readily available sequence comparison programs. These publicly and commercially available computer programs can calculate % identity between two or more sequences.

% identity may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local identity or similarity.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, the default values may be used when using such software for sequence comparisons. For example when using the GCG Wisconsin Bestfit package (see below) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Altschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). The GCG Bestfit program may be used.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). The public default values for the GCG package may be used, or in the case of other software, the default matrix, such as BLOSUM62.

Once the software has produced an optimal alignment, it is possible to calculate % homology, such as % sequence identity. The software typically does this as part of the sequence comparison and generates a numerical result.

The terms “variant” or “derivative” in relation to amino acid sequences includes any substitution of, variation of, modification of, replacement of, deletion of or addition of one (or more) amino acids from or to the sequence providing the resultant amino acid sequence retains substantially the same activity as the unmodified sequence, such as having at least the same activity as the polypeptides.

Polypeptides having the specific amino acid sequence disclosed here, or fragments or homologues thereof may be modified for use in the methods and compositions described here. Typically, modifications are made that maintain the biological activity of the sequence Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the polypeptides described here Amino acid substitutions may include the use of non-naturally occurring analogues, for example to increase blood plasma half-life of a therapeutically administered polypeptide.

### Fragments

Polypeptides for use in the methods and compositions described here also include fragments of the full length sequence of any of the polypeptides identified above. Fragments may comprise at least one epitope. Methods of identifying epitopes are well known in the art. Fragments will typically comprise at least 6 amino acids, such as at least 10, 20, 30, 50 or 100 amino acids.

Included are fragments comprising or consisting of, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145 or more residues from a relevant amino acid sequence.

We further describe peptides comprising a portion of a polypeptide as described here. Thus, fragments of and its homologues, variants or derivatives are included. The peptides may be between 2 and 200 amino acids, such as between 4 and 40 amino acids in length. The peptide may be derived from a polypeptide as disclosed here, for example by digestion with a suitable enzyme, such as trypsin. Alternatively the peptide, fragment, etc may be made by recombinant means, or synthesised synthetically.

Such fragments may be used to generate probes to preferentially detect polypeptide expression, for example, through antibodies generated against such fragments. These antibodies would be expected to bind specifically to the polypeptide, and are useful in the methods of diagnosis and treatment disclosed here.

The polypeptide and its fragments, homologues, variants and derivatives, may be made by recombinant means. However they may also be made by synthetic means using techniques well known to skilled persons such as solid phase synthesis. The proteins may also be produced as fusion proteins, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence of interest to allow removal of fusion protein sequences. The fusion protein may be one which will not hinder the function of the protein of interest sequence. Proteins may also be obtained by purification of cell extracts from animal cells.

The polypeptides, variants, homologues, fragments and derivatives disclosed here may be in a substantially isolated form. It will be understood that such polypeptides may be mixed with carriers or diluents which will not interfere with the intended purpose of the protein and still be regarded as substantially isolated. A variant, homologue, fragment or derivative may also be in a substantially purified form, in which case it will generally comprise the protein in a preparation in which more than 90%, e.g. 95%, 98% or 99% of the protein in the preparation is a protein.

The polypeptides, variants, homologues, fragments and derivatives disclosed here may be labelled with a revealing label. The revealing label may be any suitable label which allows the polypeptide, etc to be detected. Suitable labels include radioisotopes, e.g. ¹²⁵I, enzymes, antibodies, polynucleotides and linkers such as biotin. Labelled polypeptides may be used in diagnostic procedures such as immunoassays to determine the amount of a polypeptide in a sample. Polypeptides or labelled polypeptides may also be used in serological or cell-mediated immune assays for the detection of immune reactivity to said polypeptides in animals and humans using standard protocols.

The polypeptides, variants, homologues, fragments and derivatives disclosed here, optionally labelled, may also be fixed to a solid phase, for example the surface of an immunoassay well or dipstick. Such labelled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

The polypeptides, variants, homologues, fragments and derivatives disclosed here may be used in in vitro or in vivo cell culture systems to study the role of their corresponding genes and homologues thereof in cell function, including their function in disease. For example, truncated or modified polypeptides may be introduced into a cell to disrupt the normal functions which occur in the cell. The polypeptides may be introduced into the cell by in situ expression of the polypeptide from a recombinant expression vector (see below). The expression vector optionally carries an inducible promoter to control the expression of the polypeptide.

The use of appropriate host cells, such as insect cells or mammalian cells, is expected to provide for such post-translational modifications (e.g. myristolation, glycosylation, truncation, lapidation and tyrosine, serine or threonine phosphorylation) as may be needed to confer optimal biological activity on recombinant expression products. Such cell culture systems in which the polypeptides, variants, homologues, fragments and derivatives disclosed here are expressed may be used in assay systems to identify candidate substances which interfere with or enhance the functions of the polypeptides in the cell.

### Nucleic Acids

The methods and compositions described here may employ polynucleotides, nucleic acids, as well as variants, homologues, derivatives and fragments of any of these.

Nucleic acids of interest include those capable of encoding any of the polypeptides set out above, for example a nucleic acid encoding Flt-1, a nucleic acid encoding a VEGF binding domain of Flt-1, a nucleic acid encoding a membrane anchoring domain, a nucleic acid encoding a transmembrane domain of Flt1, a nucleic acid encoding an intracellular domain and a nucleic acid encoding an intracellular signalling domain of Flt-1, etc.

The terms “polynucleotide”, “nucleotide” and “nucleic acid” may be used interchangeably, and should be understood to specifically include both cDNA and genomic sequences. These terms are also intended to include a nucleic acid sequence capable of encoding a polypeptide and/or a fragment, derivative, homologue or variant of this.

Where reference is made to a Flt-1 nucleic acid, this should be taken as a reference to any member of the Flt-1 family of nucleic acids. Also included are any one or more of the nucleic acid sequences set out as “Other nucleic acid sequences” below.

For example, the Flt-1 nucleic acid may comprise a human Flt-1 sequence having GenBank Accession Number NM_002019.4.

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be DNA or RNA of genomic or synthetic or recombinant origin which may be double-stranded or single-stranded whether representing the sense or antisense strand or combinations thereof. The term nucleotide sequence may be prepared by use of recombinant DNA techniques (for example, recombinant DNA).

The term “nucleotide sequence” may means DNA.

### Other Nucleic Acids

We also provide nucleic acids which are fragments, homologues, variants or derivatives of any nucleic acid of interest.

The terms “variant”, “homologue”, “derivative” or “fragment” in relation to a nucleic acid include any substitution of, variation of, modification of, replacement of, deletion of or addition of one (or more) nucleic acids from or to the sequence of a nucleotide sequence. Unless the context admits otherwise, references to the specific nucleic acid include references to such variants, homologues, derivatives and fragments of the nucleic acid.

The resultant nucleotide sequence may encode a polypeptide having any one or more activities. The term “homologue” may be intended to cover identity with respect to structure and/or function such that the resultant nucleotide sequence encodes a polypeptide which has activity.

With respect to sequence identity (i.e. similarity), there may be at least 70%, at least 75%, at least 85% or at least 90% sequence identity. There may be at least 95%, such as at least 98%, sequence identity to a relevant sequence. These terms also encompass allelic variations of the sequences.

### Variants, Derivatives and Homologues

Nucleic acid variants, fragments, derivatives and homologues may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of this document, it is to be understood that the polynucleotides may be modified by any method available in the art. Such modifications may be carried out in order to enhance the in vivo activity or life span of polynucleotides of interest.

Where the polynucleotide is double-stranded, both strands of the duplex, either individually or in combination, are encompassed by the methods and compositions described here. Where the polynucleotide is single-stranded, it is to be understood that the complementary sequence of that polynucleotide is also included.

The terms “variant”, “homologue” or “derivative” in relation to a nucleotide sequence include any substitution of, variation of, modification of, replacement of, deletion of or addition of one (or more) nucleic acid from or to the sequence. Said variant, homologues or derivatives may code for a polypeptide having biological activity. Such fragments, homologues, variants and derivatives of nucleic acids may comprise modulated activity, as set out above.

As indicated above, with respect to sequence identity, a “homologue” may have at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to the relevant sequence. There may be at least 95% identity, at least 96% identity, at least 97% identity, at least 98% identity or at least 99% identity. Nucleotide identity comparisons may be conducted as described above. A sequence comparison program which may be used is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

### Hybridisation

We further describe nucleotide sequences that are capable of hybridising selectively to any of the sequences presented herein, or any variant, fragment or derivative thereof, or to the complement of any of the above. Nucleotide sequences may be at least 15 nucleotides in length, such as at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridising to the nucleotide sequences presented herein, or to their complement, may be at least 40% homologous, at least 45% homologous, at least 50% homologous, at least 55% homologous, at least 60% homologous, at least 65% homologous, at least 70% homologous, at least 75% homologous, at least 80% homologous, at least 85% homologous, at least 90% homologous, or at least 95% homologous to the corresponding nucleotide sequences presented herein. Such polynucleotides may be generally at least 70%, at least 80 or 90% or at least 95% or 98% homologous to the corresponding nucleotide sequences over a region of at least 20, such as at least 25 or 30, for instance at least 40, 60 or 100 or more contiguous nucleotides.

The term “selectively hybridizable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, such as less than 100 fold as intense as the specific interaction observed with the target DNA. The intensity of interaction may be measured, for example, by radiolabelling the probe, e.g. with ³²P or ³³P or with non-radioactive probes (e.g., fluorescent dyes, biotin or digoxigenin).

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained below.

Maximum stringency typically occurs at about Tm−5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

We provide nucleotide sequences that may be able to hybridise to the nucleic acids, fragments, variants, homologues or derivatives under stringent conditions (e.g. 65° C. and 0.1×SSC (1×SSC=0.15 M NaCl, 0.015 M Na₃ Citrate pH 7.0)).

### Generation of Homologues, Variants and Derivatives

Polynucleotides which are not 100% identical to the relevant sequences but which are also included, as well as homologues, variants and derivatives of the nucleic acid can be obtained in a number of ways. Other variants of the sequences may be obtained for example by probing DNA libraries made from a range of individuals, for example individuals from different populations. For example, homologues may be identified from other individuals, or other species. Further recombinant nucleic acids and polypeptides may be produced by identifying corresponding positions in the homologues, and synthesising or producing the molecule as described elsewhere in this document.

In addition, other viral/bacterial, or cellular homologues of a nucleic acid, particularly cellular homologues found in mammalian cells (e.g. rat, mouse, bovine and primate cells), may be obtained and such homologues and fragments thereof in general will be capable of selectively hybridising to human nucleic acid. Such homologues may be used to design non-human nucleic acids, fragments, variants and homologues. Mutagenesis may be carried out by means known in the art to produce further variety.

Sequences of nucleic acid homologues may be obtained by probing cDNA libraries made from or genomic DNA libraries from other animal species, and probing such libraries with probes comprising all or part of any of the nucleic acids, fragments, variants and homologues, or other fragments of the nucleic acid under conditions of medium to high stringency.

Similar considerations apply to obtaining species homologues and allelic variants of the polypeptide or nucleotide sequences disclosed here.

Variants and strain/species homologues may also be obtained using degenerate PCR which will use primers designed to target sequences within the variants and homologues encoding conserved amino acid sequences within the sequences of the nucleic acids. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labelled fragments the nucleic acid sequences.

In addition, homologous sequences may be identified by searching nucleotide and/or protein databases using search algorithms such as the BLAST suite of programs.

Alternatively, such polynucleotides may be obtained by site directed mutagenesis of characterised sequences, for example, nucleic acids, or variants, homologues, derivatives or fragments thereof. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The polynucleotides described here may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 8, 9, 10, or 15, such as at least 20, for example at least 25, 30 or 40 nucleotides in length, and are also encompassed by the term “polynucleotides” as used herein.

Polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Primers comprising fragments of a nucleic acid are particularly useful in the methods of detection of nucleic acid expression, such as up-regulation of nucleic acid expression. Suitable primers for amplification of nucleic acids may be generated from any suitable stretch of the nucleic acid. Primers which may be used include those capable of amplifying a sequence of a nucleic acid which is specific.

Although nucleic acid primers may be provided on their own, they are most usefully provided as primer pairs, comprising a forward primer and a reverse primer.

Longer polynucleotides will generally be produced using recombinant means, for example using a PCR (polymerase chain reaction) cloning techniques. This will involve making a pair of primers (e.g. of about 15 to 30 nucleotides), bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector

Polynucleotides or primers may carry a revealing label. Suitable labels include radioisotopes such as ³²P or ³⁵S, digoxigenin, fluorescent dyes, enzyme labels, or other protein labels such as biotin. Such labels may be added to polynucleotides or primers and may be detected using by techniques known per se. Polynucleotides or primers or fragments thereof labelled or unlabeled may be used by a person skilled in the art in nucleic acid-based tests for detecting or sequencing polynucleotides in the human or animal body.

Such tests for detecting generally comprise bringing a biological sample containing DNA or RNA into contact with a probe comprising a polynucleotide or primer under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected. Suitable assay methods of this and other formats can be found in for example WO89/03891 and WO90/13667.

Tests for sequencing nucleotides, for example, the nucleic acids, involve bringing a biological sample containing target DNA or RNA into contact with a probe comprising a polynucleotide or primer under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

### Control Regions

For some purposes, it may be necessary to utilise or investigate control regions of a nucleic acid such as Flt-1. Such control regions include promoters, enhancers and locus control regions. By a control region we mean a nucleic acid sequence or structure which is capable of modulating the expression of a coding sequence which is operatively linked to it.

For example, control regions are useful in generating transgenic animals expressing Flt-1. Furthermore, control regions may be used to generate expression constructs for Flt-1. This is described in further detail below.

Identification of control regions of Flt-1 is straightforward, and may be carried out in a number of ways. For example, the coding sequence of Flt-1 may be obtained from an organism, by screening a cDNA library using a human or mouse Flt-1 cDNA sequence as a probe. 5′ sequences may be obtained by screening an appropriate genomic library, or by primer extension as known in the art. Database searching of genome databases may also be employed. Such 5′ sequences which are particularly of interest include non-coding regions. The 5′ regions may be examined by eye, or with the aid of computer programs, to identify sequence motifs which indicate the presence of promoter and/or enhancer regions.

Furthermore, sequence alignments may be conducted of Flt-1 nucleic acid sequences from two or more organisms. By aligning Flt-1 sequences from different species, it is possible to determine which regions of the amino acid sequence are conserved between different species. Such conserved regions are likely to contain control regions for the gene in question (i.e., Flt-1). The mouse and human genomic sequences as disclosed here, for example, a mouse Flt-1 genomic sequence, may be employed for this purpose. Furthermore, Flt-1 homologues from other organisms may be obtained using standard methods of screening using appropriate probes generated from the mouse and human Flt-1 sequences. The genome of the pufferfish (Takifugu rubripes) or zebrafish may also be screened to identify a Flt-1 homologue. Comparison of the 5′ non-coding region of the Fugu or zebrafish Flt-1 gene with a mouse or human genomic Flt-1 sequence may be used to identify conserved regions containing control regions.

Deletion studies may also be conducted to identify promoter and/or enhancer regions for Flt-1.

The identity of putative control regions may be confirmed by molecular biology experiments, in which the candidate sequences are linked to a reporter gene and the expression of the reporter detected.

### Further Aspects

Further aspects and embodiments of the invention are now set out in the following numbered Paragraphs; it is to be understood that the invention encompasses these aspects:

Paragraph 1. A membrane-bound Flt-1 decoy which has its intracellular signalling domain removed.

Paragraph 2. A mirtron capable of targeting VEGF delivered as a genetic construct using AAV2 or AAV-DJ.

### EXAMPLES

### Example 1. Materials and Methods: Cell Culture

All experiments were conducted in HEK293T cells cultivated in DMEM supplemented with 10% Fetal Bovine Serum and 1% Penicillin/Streptomycin.

All experiments were performed in 24 well plates seeded at 5×10⁴ cells per well.

If transfected, pcoFlt1, pcoFlt1-Mirt or control peGFP-C1 were transfected at 500 ng per well while psiCheck2.2-VegfT (Kock et al NAR 2015) was transfected at 250 ng per well using Lipofectamine 2000 (ThermoFisher Scientific) as per manufacturer's instructions.

### Example 2. Materials and Methods: Plasmid Construction—pcoFlt1

pcoFlt1 was constructed with a codon-optimized truncated version of human Flt1 gene (first 2454 nucleotides of PubMed accession number NM_002019; Sequence attached in Table D1 below) with a 3′ HA tag ordered from IDT.

The gene was amplified by PCR using these 2 primers (5′-acaGCTAGCACCATGGTTTCTTACTGGGACACGGG-3′ (SEQ ID NO: 7) and 5′-atcGGATCCTCATTAAGCGTAGTCTGGAACGTCATA-3′) (SEQ ID NO: 8) and cloned into peGFP-C1 between NheI and BamHI sites.

TABLE D1
coFlt1 sequence (SEQ ID NO: 9)

| | |
|---|---|
| coFlt1 | GCTAGCACCATGGTTTCTTACTGGGACACGGGGGTACTCCTTTGCGCAC |
| | TTCTGTCTTGTTTGCTTCTGACGGGGTCCTCTTCTGGGTCTAAGCTGAA |
| | AGATCCGGAGCTGTCTCTGAAGGGCACGCAACACATCATGCAAGCTGGT |
| | CAAACTTTGCATCTCCAGTGCCGGGGGGAGGCTGCGCATAAGTGGTCTT |
| | TGCCTGAAATGGTATCAAAGGAGTCCGAACGCCTCAGTATAACTAAAAG |
| | CGCATGTGGTCGGAACGGAAAACAATTTTGTAGCACGCTCACACTCAAT |
| | ACTGCTCAAGCCAATCATACCGGATTTTACTCTTGCAAATATCTTGCGG |
| | TACCGACCTCTAAAAAGAAAGAAACCGAAAGCGCCATCTACATCTTCAT |
| | TTCTGACACAGGCCGGCCATTTGTTGAAATGTATTCAGAGATCCCTGAA |
| | ATCATCCACATGACTGAGGGGCGAGAACTTGTTATACCCTGCCGGGTCA |
| | CCAGTCCCAACATTACGGTGACCCTCAAAAAATTCCCACTGGATACGCT |
| | TATCCCGGACGGGAAGCGCATTATATGGGACTCTCGAAAAGGGTTTATC |
| | ATTTCAAATGCCACGTACAAAGAAATCGGTCTGCTGACCTGCGAGGCCA |
| | CGGTGAATGGCCACTTGTATAAGACTAATTACCTCACTCACCGCCAGAC |
| | AAACACAATTATCGATGTACAGATCTCCACACCTCGACCCGTTAAGCTG |
| | CTCAGAGGGCATACACTTGTACTTAACTGCACAGCCACCACCCCGCTGA |
| | ATACGAGAGTACAGATGACCTGGTCATATCCGGACGAGAAGAACAAAAG |
| | AGCTTCCGTGCGAAGGCGAATCGACCAGTCAAACTCCCATGCCAACATT |
| | TTCTACTCTGTTCTGACGATCGACAAAATGCAGAACAAAGATAAGGGTT |
| | TGTACACTTGTCGAGTCCGGAGTGGTCCATCTTTCAAAAGTGTAAATAC |
| | TTCAGTGCATATCTACGATAAAGCCTTTATTACGGTCAAGCATCGAAAG |
| | CAACAAGTACTCGAAACTGTAGCAGGGAAACGCTCCTACCGGTTGTCTA |
| | TGAAGGTAAAGGCTTTTCCCAGCCCCGAAGTAGTGTGGCTCAAAGATGG |
| | GCTTCCGGCGACGGAGAAGAGCGCTAGGTACTTGACAAGGGGCTACTCA |
| | CTCATAATAAAGGACGTGACGGAAGAGGACGCGGGGAATTATACAATAC |
| | TTTTGTCCATAAAACAATCTAACGTTTTCAAAAACCTCACGGCGACTTT |
| | GATTGTCAATGTGAAACCTCAAATCTACGAGAAAGCTGTCTCTTCCTTC |
| | CCGGACCCAGCGTTGTATCCACTCGGATCTAGGCAGATTCTCACCTGTA |
| | CAGCCTACGGGATACCGCAGCCTACTATTAAATGGTTTTGGCACCCATG |
| | TAACCATAACCACTCAGAAGCTCGCTGCGATTTCTGCTCTAATAATGAA |
| | GAGAGTTTCATACTTGACGCGGATTCCAACATGGGAAACCGCATTGAGT |
| | CAATTACCCAACGGATGGCAATCATCGAAGGGAAGAACAAGATGGCCTC |
| | AACTCTCGTGGTAGCAGATAGCCGAATTTCAGGAATATACATTTGTATC |
| | GCGTCTAATAAGGTAGGAACTGTCGGCCGAAATATATCCTTTTACATCA |
| | CGGATGTCCCCAACGGATTTCATGTAAATCTGGAAAAGATGCCCACAGA |
| | AGGAGAGGATCTGAAACTTTCCTGTACGGTAAATAAGTTCCTCTATCGC |
| | GACGTAACATGGATTTTGCTCCGGACCGTTAACAACCGCACCATGCATT |
| | ACAGTATATCTAAGCAGAAGATGGCCATTACTAAAGAGCATTCTATTAC |
| | ACTGAACCTCACTATCATGAATGTATCTCTTCAGGATAGTGGCACGTAC |
| | GCGTGTCGCGCTAGGAATGTGTATACTGGCGAGGAAATACTCCAGAAGA |
| | AAGAGATTACGATCAGGGACCAGGAGGCACCATACCTCCTGAGAAACCT |
| | TTCTGACCACACGGTGGCCATAAGTAGTAGTACGACACTTGATTGCCAT |
| | GCGAACGGTGTTCCGGAACCACAGATCACATGGTTTAAGAACAATCACA |
| | AAATCCAGCAGGAGCCCGGGATCATACTTGGACCTGGGAGCTCCACGTT |
| | GTTTATTGAAAGGGTTACCGAAGAGGACGAAGGGGTCTATCATTGTAAG |
| | GCAACAAATCAAAAGGGATCAGTTGAAAGTAGTGCATACTTGACCGTGC |
| | AAGGAACTAGTGATAAATCCAACCTTGAGCTGATTACGTTGACGTGCAC |
| | GTGCGTAGCAGCTACCTTGTTCTGGCTGCTCTTGACCCTGTTCATTCGG |
| | AAAATGAAAAGGTCCTCTAGTGAGATAAAAACTGACTACCTTTCCATAA |
| | TAATGGACCCGGACGAAGTTCCACTGGACGAACAGTGTGAACGCCTGCC |
| | GTACGACGCGTCCTATCCTTATGACGTTCCAGACTACGCTTAATGAGGA |
| | TCC |

### Example 3. Materials and Methods: Plasmid Construction—pscoFlt1

pscoFlt1 (Table D2) was designed to be similar to the version described in Kong H L et al (Human Gene Therapy 1998), and was constructed by linear amplification of pcoFlt1 with these 2 primers (5′-ACAGGATCCTAGTGATGGTGGTGATGATGGCCTCCCTTAACGGGTCGAGGTGTGGAG-3′ (SEQ ID NO: 10) and 5′-GCGTCCTATCCTTATGACGTTCC-3′) (SEQ ID NO: 11) to eliminate the 3′ end of coFlt1 by cutting with BamHI and annealing using the BamHI sites.

TABLE D2
scoFlt1 sequence (SEQ ID NO: 12)

| | |
|---|---|
| scoFlt1 | GCTAGCACCATGGTTTCTTACTGGGACACGGGGGTACTCCTTTGCGCAC |
| | TTCTGTCTTGTTTGCTTCTGACGGGGTCCTCTTCTGGGTCTAAGCTGAA |
| | AGATCCGGAGCTGTCTCTGAAGGGCACGCAACACATCATGCAAGCTGGT |
| | CAAACTTTGCATCTCCAGTGCCGGGGGGAGGCTGCGCATAAGTGGTCTT |
| | TGCCTGAAATGGTATCAAAGGAGTCCGAACGCCTCAGTATAACTAAAAG |
| | CGCATGTGGTCGGAACGGAAAACAATTTTGTAGCACGCTCACACTCAAT |
| | ACTGCTCAAGCCAATCATACCGGATTTTACTCTTGCAAATATCTTGCGG |
| | TACCGACCTCTAAAAAGAAAGAAACCGAAAGCGCCATCTACATCTTCAT |
| | TTCTGACACAGGCCGGCCATTTGTTGAAATGTATTCAGAGATCCCTGAA |
| | ATCATCCACATGACTGAGGGGCGAGAACTTGTTATACCCTGCCGGGTCA |
| | CCAGTCCCAACATTACGGTGACCCTCAAAAAATTCCCACTGGATACGCT |
| | TATCCCGGACGGGAAGCGCATTATATGGGACTCTCGAAAAGGGTTTATC |
| | ATTTCAAATGCCACGTACAAAGAAATCGGTCTGCTGACCTGCGAGGCCA |
| | CGGTGAATGGCCACTTGTATAAGACTAATTACCTCACTCACCGCCAGAC |
| | AAACACAATTATCGATGTACAGATCTCCACACCTCGACCCGTTAAGGGA |
| | GGCCATCATCACCACCATCACTAGGATCC |

### Example 4. Materials and Methods: Plasmid Construction—coFlt1-Mirt

The 2 mirtrons were inserted into pcoFlt1 the same way, using linear PCR amplification of the entire plasmid with additional mirtron sequences on each end, then using BbsI sites to produce corresponding overhangs for annealing.

The sequences are shown in Table D3.

TABLE D3
coFlt1-Mirt sequence (SEQ ID NO: 13)

| | |
|---|---|
| coFlt1-Mirt | GCTAGCACCATGGTTTCTTACTGGGACACGGGGGTACTCCTTTGCGCAC |
| (mirtrons | TTCTGTCTTGTTTGCTTCTGACGGGGTCCTCTTCTGGGTCTAAGCTGAA |
| highlighted | AGATCCGGAGCTGTCTCTGAAGGTAAATGTATGTATGTGGGTGTTCAAG |
| in grey) | AGACACCCACACACATACATCTCAGTTTTTTCTCTTTCTTTCAGGGCAC |
| | GCAACACATCATGCAAGCTGGTCAAACTTTGCATCTCCAGTGCCGGGGG |
| | GAGGCTGCGCATAAGTGGTCTTTGCCTGAAATGGTATCAAAGGTAGATT |
| | ATGCGGATTAAATTTCAAGAGAGTTTGATCCGCATAATCTGTCAGTTTT |
| | TTCTCTTTCTTTCAGGAGTCCGAACGCCTCAGTATAACTAAAAGCGCAT |
| | GTGGTCGGAACGGAAAACAATTTTGTAGCACGCTCACACTCAATACTGC |
| | TCAAGCCAATCATACCGGATTTTACTCTTGCAAATATCTTGCGGTACCG |
| | ACCTCTAAAAAGAAAGAAACCGAAAGCGCCATCTACATCTTCATTTCTG |
| | ACACAGGCCGGCCATTTGTTGAAATGTATTCAGAGATCCCTGAAATCAT |
| | CCACATGACTGAGGGGCGAGAACTTGTTATACCCTGCCGGGTCACCAGT |
| | CCCAACATTACGGTGACCCTCAAAAAATTCCCACTGGATACGCTTATCC |
| | CGGACGGGAAGCGCATTATATGGGACTCTCGAAAAGGGTTTATCATTTC |
| | AAATGCCACGTACAAAGAAATCGGTCTGCTGACCTGCGAGGCCACGGTG |
| | AATGGCCACTTGTATAAGACTAATTACCTCACTCACCGCCAGACAAACA |
| | CAATTATCGATGTACAGATCTCCACACCTCGACCCGTTAAGCTGCTCAG |
| | AGGGCATACACTTGTACTTAACTGCACAGCCACCACCCCGCTGAATACG |
| | AGAGTACAGATGACCTGGTCATATCCGGACGAGAAGAACAAAAGAGCTT |
| | CCGTGCGAAGGCGAATCGACCAGTCAAACTCCCATGCCAACATTTTCTA |
| | CTCTGTTCTGACGATCGACAAAATGCAGAACAAAGATAAGGGTTTGTAC |
| | ACTTGTCGAGTCCGGAGTGGTCCATCTTTCAAAAGTGTAAATACTTCAG |
| | TGCATATCTACGATAAAGCCTTTATTACGGTCAAGCATCGAAAGCAACA |
| | AGTACTCGAAACTGTAGCAGGGAAACGCTCCTACCGGTTGTCTATGAAG |
| | GTAAAGGCTTTTCCCAGCCCCGAAGTAGTGTGGCTCAAAGATGGGCTTC |
| | CGGCGACGGAGAAGAGCGCTAGGTACTTGACAAGGGGCTACTCACTCAT |
| | AATAAAGGACGTGACGGAAGAGGACGCGGGGAATTATACAATACTTTTG |
| | TCCATAAAACAATCTAACGTTTTCAAAAACCTCACGGCGACTTTGATTG |
| | TCAATGTGAAACCTCAAATCTACGAGAAAGCTGTCTCTTCCTTCCCGGA |
| | CCCAGCGTTGTATCCACTCGGATCTAGGCAGATTCTCACCTGTACAGCC |
| | TACGGGATACCGCAGCCTACTATTAAATGGTTTTGGCACCCATGTAACC |
| | ATAACCACTCAGAAGCTCGCTGCGATTTCTGCTCTAATAATGAAGAGAG |
| | TTTCATACTTGACGCGGATTCCAACATGGGAAACCGCATTGAGTCAATT |
| | ACCCAACGGATGGCAATCATCGAAGGGAAGAACAAGATGGCCTCAACTC |
| | TCGTGGTAGCAGATAGCCGAATTTCAGGAATATACATTTGTATCGCGTC |
| | TAATAAGGTAGGAACTGTCGGCCGAAATATATCCTTTTACATCACGGAT |
| | GTCCCCAACGGATTTCATGTAAATCTGGAAAAGATGCCCACAGAAGGAG |
| | AGGATCTGAAACTTTCCTGTACGGTAAATAAGTTCCTCTATCGCGACGT |
| | AACATGGATTTTGCTCCGGACCGTTAACAACCGCACCATGCATTACAGT |
| | ATATCTAAGCAGAAGATGGCCATTACTAAAGAGCATTCTATTACACTGA |
| | ACCTCACTATCATGAATGTATCTCTTCAGGATAGTGGCACGTACGCGTG |
| | TCGCGCTAGGAATGTGTATACTGGCGAGGAAATACTCCAGAAGAAAGAG |
| | ATTACGATCAGGGACCAGGAGGCACCATACCTCCTGAGAAACCTTTCTG |
| | ACCACACGGTGGCCATAAGTAGTAGTACGACACTTGATTGCCATGCGAA |
| | CGGTGTTCCGGAACCACAGATCACATGGTTTAAGAACAATCACAAAATC |
| | CAGCAGGAGCCCGGGATCATACTTGGACCTGGGAGCTCCACGTTGTTTA |
| | TTGAAAGGGTTACCGAAGAGGACGAAGGGGTCTATCATTGTAAGGCAAC |
| | AAATCAAAAGGGATCAGTTGAAAGTAGTGCATACTTGACCGTGCAAGGA |
| | ACTAGTGATAAATCCAACCTTGAGCTGATTACGTTGACGTGCACGTGCG |
| | TAGCAGCTACCTTGTTCTGGCTGCTCTTGACCCTGTTCATTCGGAAAAT |
| | GAAAAGGTCCTCTAGTGAGATAAAAACTGACTACCTTTCCATAATAATG |
| | GACCCGGACGAAGTTCCACTGGACGAACAGTGTGAACGCCTGCCGTACG |
| | ACGCGTCCTATCCTTATGACGTTCCAGACTACGCTTAATGAGGATCC |

TABLE D4
Primer sequences

| | |
|---|---|
| NheI coFlt1 F (SEQ ID | acaGCTAGCACCATGGTTTCTTACTGGGACACGGG |
| NO: 14) | |
| BamHI coFlt1 R (SEQ ID | atcGGATCCTCATTAAGCGTAGTCTGGAACGTCATA |
| NO: 15) | |
| sco-Flt1 F | GCGTCCTATCCTTATGACGTTCC |
| (SEQ ID NO: 16) | |
| BamHI sco-Flt1-His R | ACAGGATCCTAGTGATGGTGGTGATGATGGCCTCCCTT |
| (SEQUENCE ID NO. 17) | AACGGGTCGAGGTGTGGAG |
| Bbs1 | tatGAAGACTAGGTGTCTCTTGAACACCCACATACATA |
| CoFlt1 Mirt 1 R (SEQ | CATTTACCTTCAGAGACAGCTCCGGATC |
| ID NO: 18) | |
| Bbs1 CoFlt1 Mirt 1 F | tatGAAGACTACACCCACACACATACATCTCAGTTTTT |
| (SEQ ID NO: 19) | TCTCTTTCTTTCAGGGCACGCAACACATCATGGTATC |
| BbsI CoFlt1 Mirt 8 R | tGAAGACTACTTGAAATTTAATCCGCATAATCTACCTT |
| (SEQ ID NO: 20) | TGATACCATTTCAGGCAAAGAC |
| BbsI CoFlt1 Mirt 8 F | atGAAGACTACAAGAGAGTTTGATCCGCATAATCTGTC |
| (SEQ ID NO: 21) | AGTTTTTTCTCTTTCTTTCAGGAGTCCGAACGCCTCAG |
| | TATAA |
| VEGFA qPCR F | CGGATCAAACCTCACCAAGGC |
| (SEQ ID NO: 22) | |
| VEGFA qPCR R | AGGGAGGCTCCTTCCTCCT |
| (SEQ ID NO: 23) | |
| GAPDH qPCR F | aaggtgaaggtcggagtcaa |
| (SEQ ID NO: 24) | |
| GAPDH qPCR R | gaagatggtgatgggatttc |
| (SEQ ID NO: 25) | |

### Example 3. Materials and Methods: VEGF Depletion

2 days after transfection of 500 ng of pcoFlt1, pcoFlt1-Mirt, pscoFlt1 or peGFP-C1 into HEK293 cells in 24 well plate format seeded with ˜100000 cells, 500 μl of fresh medium was added with 2 ng/ml of recombinant VEGF (Lonza).

2 hours after addition of fresh medium, 200 μl of medium was removed for ELISA (RnD Systems VEGF Quantikine ELISA kit) as per manufacturer's instructions.

For pscoFlt1, as the soluble Flt1 is not expressed on the cells, to remove bound VEGF, the medium is incubated with 80 μl of HisPur Ni-NTA resin (ThermoFisher Scientific) for 30 mins at 4° C. before ELISA.

### Example 4. Materials and Methods: VEGF Knockdown Using Dual Luciferase Assay

2 days after transfection with target and construct, the cells were harvested with passive lysis buffer (Promega) and assayed with Dual Luciferase Reporter Assay System (Promega) as per manufacturer's instructions.

### Example 5. Results: Membrane-Bound Flt-1 Decoy

The first technical innovation involves the realization that a soluble decoy for VEGF (i.e., a membrane-bound Flt-1 decoy) would be inferior to a membrane-bound version.

The membrane-bound Flt-1 decoy has three main benefits.
First, the membrane-bound Flt-1 decoy would be more localized to the injection site, reducing side effects associated with VEGF sequestration elsewhere.
Second, the membrane-bound Flt-1 decoy requires lower concentrations of decoys for dimerization and effective VEGF binding because the decoy's movement has fewer degrees of freedom.
Third, the membrane-bound Flt-1 decoy is expressed at high concentration on the membrane surface rather than soluble in a large extracellular volume, which increases dimerization and thus can sequester VEGF more effectively.

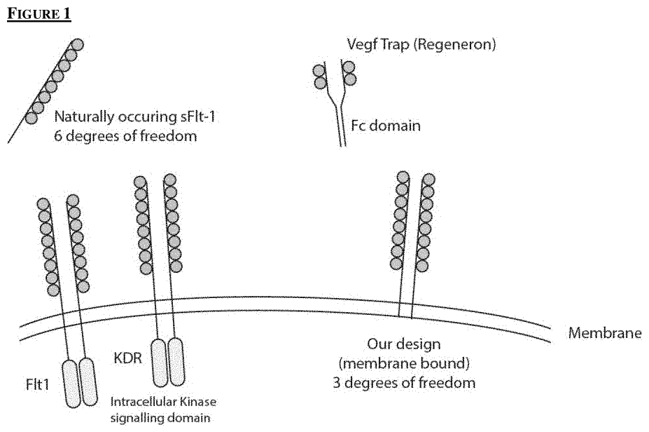

As the full length Flt1 would have an intracellular signalling domain that can transduce a signal through the SHC-GRB2, PI3K and PLC pathways that are common to multiple receptors, which makes it unwise to use, we proceeded to truncate the intracellular signalling domain away. This leaves only the transmembrane and extracellular domain of Flt-1 (see FIG. 1).

The truncated version of Flt-1 allows sequestration of VEGF without the intracellular signalling component.

### Example 6. Results: Membrane-Bound Flt-1 Decoy Depletes Exogenous VEGF

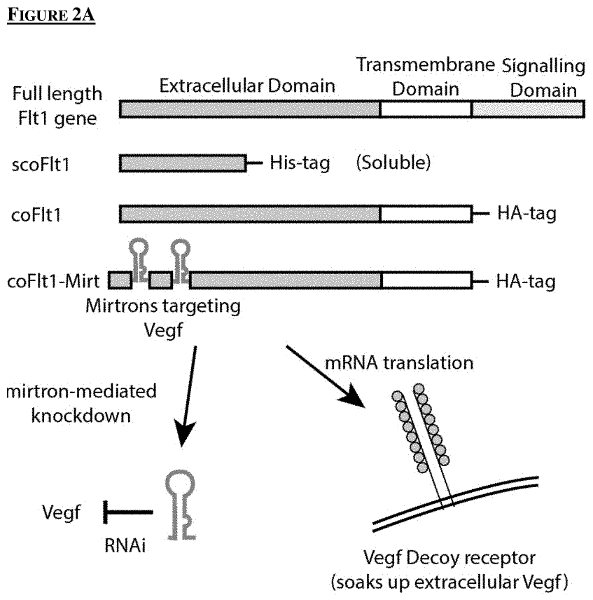

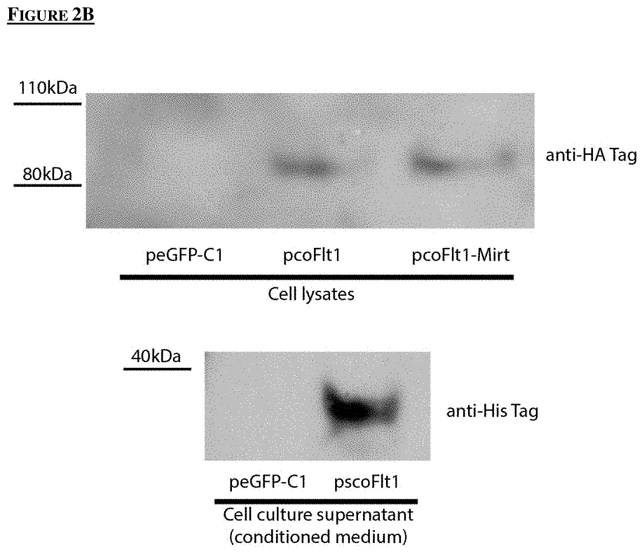

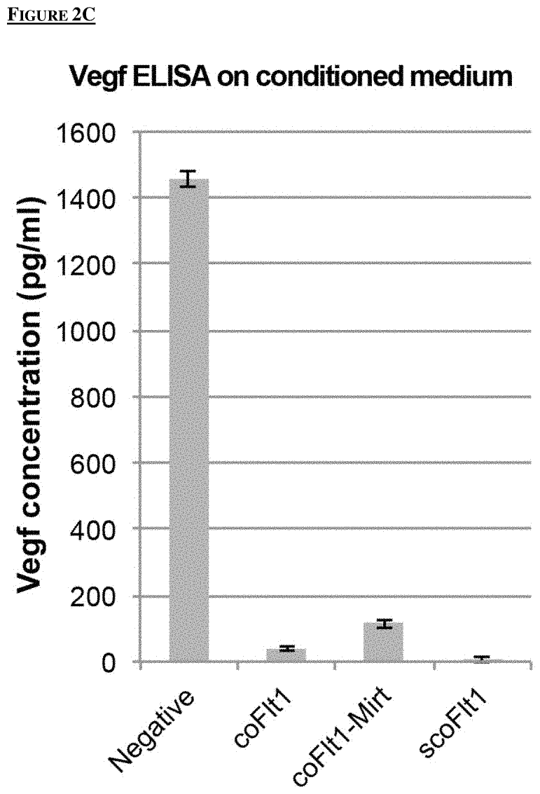

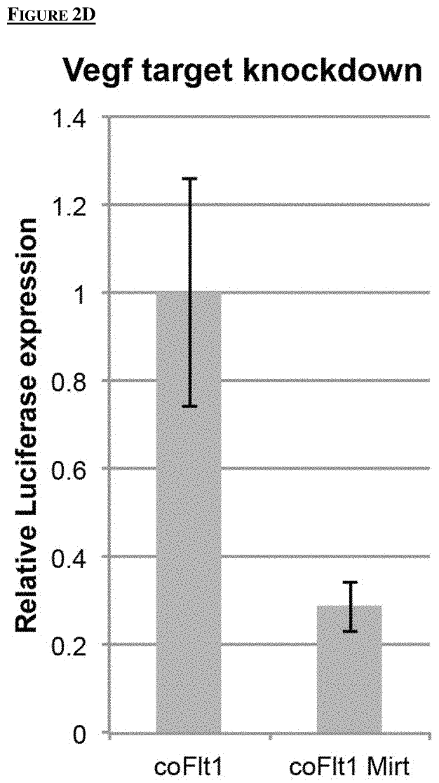

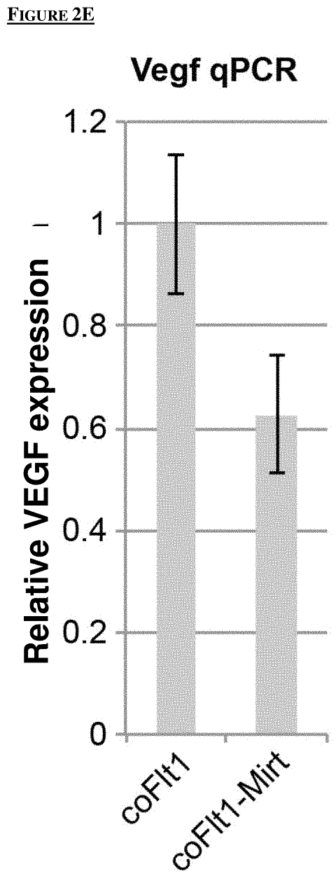

When pcoFlt1 and pcoFlt1-Mirt were transfected into HEK293T cells, the HA-tagged coFlt1 (FIG. 2A) is effectively expressed and can be detected with an anti-HA tag Western blot (FIG. 2B).

2 days after transfection, HEK293T cells expressing coFlt1 were incubated with freshly added exogenous VEGF for 2 hours at a physiologically relevant concentration of 2 ng/ml.

Medium recovered after 2 hours showed almost no decrease in VEGF concentration when HEK293T cells expressed eGFP.

On the other hand, VEGF was almost completely sequestered by coFlt1 (FIG. 2C) onto the HEK293T cells.

This demonstrates that the membrane-bound Flt-1 decoy coFlt1 can effectively sequester VEGF in extracellular fluids.

### Example 6. Results: Further Reduction of Expression of VEGF Using mMirtrons

The second technical innovation was to improve the efficacy of the therapy with an RNAi modality to reduce expression of VEGF on top of sequestration.

To this end, we added the 2 VEGF mirtrons into the coding region of coFlt1 as two separate introns (coFlt1-Mirt) (FIG. 2A). The VEGF mirtrons are RNAi effectors encoded as introns we had previously invented (Kock et al, NAR 2015).

The resulting construct is referred to as coFlt1-Mirt.

We ascertained that the addition of the 2 mirtrons did not affect the expression of the membrane-bound Flt-1 decoy coFlt1 (FIG. 2B) or the sequestration efficiency (FIG. 2C) of the decoy.

Moreover, coFlt1-Mirt was also effective at reducing luciferase reporters containing VEGF targeting sequences in the 3′ UTR (FIG. 2D). This luciferase reporter plasmid contains a Firefly luciferase for transfection normalization and a Renilla luciferase that contains the target VEGF sequences in the 3′UTR of the gene.

Successful RNAi against the target sequence will reduce Renilla luciferase expression relative to firefly luciferase expression.

To demonstrate that coFlt1-Mirt could also reduce endogenous VEGF production and target endogenous sequences rather than target sequences put into reporter genes, we stimulated a hypoxic response by adding 300 μM of cobalt chloride for 2 days to induce VEGF expression in coFlt1 or coFlt1-Mirt transfected HEK293T cells.

Using quantitative RT-PCR (see Table 1 for qPCR primer sequences), VEGF mRNA expression normalised to GAPDH expression was markedly reduced by coFlt1-Mirt (FIG. 2E) compared to coFlt1 only.

As the difference between the two plasmids is simply the presence of the 2 mirtrons, this suggests that the combination of therapies worked individually in the coFlt1-Mirt construct.

Thus, we demonstrate that our dual component construct can effectively (a) sequester exogenous VEGF through the membrane-bound VEGF decoy coFlt1 and (b) reduce endogenous VEGF production through mirtrons within the coFlt1 construct.

### REFERENCES

Heier J S, Kherani S, Desai S, Dugel P, Kaushal S, Cheng S H, Intravitreous injection of AAV2-sFLT01 in patients with advanced neovascular age-related macular degeneration: a phase 1, open-label trial. 390, 50-61 (2017).
Constable I J, Pierce C M, Lai C M, Magno A L, Degli-Esposti M A, French M A Phase 2a Randomized Clinical Trial: Safety and Post Hoc Analysis of Subretinal rAAV.sFLT-1 for Wet Age-related Macular Degeneration. 14, 168-175 (2016).
Kock K H, Kong K W, Hoon S, Seow Y. Functional VEGFA knockdown with artificial 3′-tailed mirtrons defined by 5′ splice site and branch point. 43, 6568-78 (2015).
Kong H L, Hecht D, Song W, Kovesdi I, Hackett N R, Yayon A, Crystal R G. Regional suppression of tumor growth by in vivo transfer of a cDNA encoding a secreted form of the extracellular domain of the flt-1 vascular endothelial growth factor receptor. 9, 823-33 (1998).
1. Ambati, B. K., Nozaki, M., Singh, N., Takeda, A., Jani, P. D., Suthar, T., Albuquerque, R. J. C., Richter, E., Sakurai, E., Newcomb, M. T., Kleinman, M. E., Caldwell, R. B., and 25 others. Corneal avascularity is due to soluble VEGF receptor-1. Nature 443: 993-997, 2006.
2. Autiero, M., Waltenberger, J., Communi, D., Kranz, A., Moons, L., Lambrechts, D., Kroll, J., Plaisance, S., De Mol, M., Bono, F., Kliche, S., Fellbrich, G., and 16 others. Role of PlGF in the intra- and intermolecular cross talk between the VEGF receptors Flt1 and Flk1. Nature Med. 9: 936-943, 2003.
3. De Bandt, M., Ben Mandi, M. H., Ollivier, V., Grossin, M., Dupuis, M., Gaudry, M., Bohlen, P., Lipson, K. E., Rice, A., Wu, Y., Gougerot-Pocidalo, M.-A., Pasquier, C. Blockade of vascular endothelial growth factor receptor I (VEGF-RI), but not VEGF-RII, suppresses joint destruction in the K/B×N model of rheumatoid arthritis. J. Immun 171: 4853-4859, 2003.
4. Fong, G.-H., Rossant, J., Breitman, M. L. Role of the Flt-1 receptor tyrosine kinase in regulating the assembly of vascular endothelium. Nature 376: 66-69, 1995.
5. He, Y., Smith, S. K., Day, K. A., Clark, D. E., Licence, D. R., Charnock-Jones, D. S. Alternative splicing of vascular endothelial growth factor (VEGF)-R1 (FLT-1) pre-mRNA is important for the regulation of VEGF activity. Molec. Endocr. 13: 537-545, 1999. 6. Imbert, A., Rosnet, O., Marchetto, S., Ollendorff, V., Birnbaum, D., Pebusque, M.-J. Characterization of a yeast artificial chromosome from human chromosome band 13q12 containing the FLT1 and FLT3 receptor-type tyrosine kinase genes. Cytogenet. Cell Genet. 67: 175-177, 1994.
7. Kaplan, R. N., Riba, R. D., Zacharoulis, S., Bramley, A. H., Vincent, L., Costa, C., MacDonald, D. D., Jin, D. K., Shido, K., Kerns, S. A., Zhu, Z., Hicklin, D., and 9 others. VEGFR1-positive haematopoietic bone marrow progenitors initiate the pre-metastatic niche. Nature 438: 820-827, 2005.
8. Kendall, R. L., Thomas, K. A. Inhibition of vascular endothelial cell growth factor activity by an endogenously encoded soluble receptor. Proc. Nat. Acad. Sci. 90: 10705-10709, 1993.
9. Kendall, R. L., Wang, G., Thomas, K. A. Identification of a natural soluble form of the vascular endothelial growth factor receptor, FLT-1, and its heterodimerization with KDR. Biochem. Biophys. Res. Commun. 226: 324-328, 1996.
10. Kleinman, M. E., Yamada, K., Takeda, A., Chandrasekaran, V., Nozaki, M., Baffi, J. Z., Albuquerque, R. J. C., Yamasaki, S., Itaya, M., Pan, Y., Appukuttan, B., Gibbs, D., and 10 others. Sequence- and target-independent angiogenesis suppression by siRNA via TLR3. Nature 452: 591-597, 2008.
11. LeCouter, J., Moritz, D. R., Li, B., Phillips, G. L., Liang, X. H., Gerber, H.-P., Hillan, K. J., Ferrara, N. Angiogenesis-independent endothelial protection of liver: role of VEGFR-1. Science 299: 890-893, 2003.
12. Levine, R. J., Maynard, S. E., Qian, C., Lim, K.-H., England, L. J., Yu, K. F., Schisterman, E. F., Thadhani, R., Sachs, B. P., Epstein, F. H, Sibai, B. M., Sukhatme, V. P., Karumanchi, S. A. Circulating angiogenic factors and the risk of preeclampsia. New Eng. J. Med. 350: 672-683, 2004.
13. Liu, Z., Hou, P., Ji, M., H., Studeman, K., Jensen, K, Vasko, V., El-Naggar, A. K., Xing, M. Highly prevalent genetic alterations in receptor tyrosine kinases and phosphatidylinositol 3-kinase/Akt and mitogen-activated protein kinase pathways in anaplastic and follicular thyroid cancers. J. Clin. Endocr. Metab. 93: 3106-3116, 2008.
14. Maynard, S. E., MM, J.-Y., Merchan, J., Lim, K.-H., Li, J., Mondal, S., Libermann, T. A., Morgan, J. P., Sellke, F. W., Stillman, I. E., Epstein, F. H., Sukhatme, V. P., Karumanchi, S. A. Excess placental soluble fins-like tyrosine kinase 1 (sFlt1) may contribute to endothelial dysfunction, hypertension, and proteinuria in preeclampsia. J. Clin. Invest. 111: 649-658, 2003.
15. Muehlenbachs, A., Fried, M., Lachowitzer, J., Mutabingwa, T. K., Duffy, P. E. Natural selection of FLT1 alleles and their association with malaria resistance in utero. Proc. Nat. Acad. Sci. 105: 14488-14491, 2008.
16. Niida, S., Kondo, T., Hiratsuka, S., Hayashi, S.-I., Amizuka, N., Noda, T., Ikeda, K., Shibuya, M. VEGF receptor 1 signalling is essential for osteoclast development and bone marrow formation in colony-stimulating factor 1-deficient mice. Proc. Nat. Acad. Sci. 102: 14016-14021, 2005.
17. Nozaki, M., Sakurai, E., Raisler, B. J., Baffi, J. Z., Witta, J., Ogura, Y., Brekken, R. A., Sage, E. H., Ambati, B. K., Ambati, J. Loss of SPARC-mediated VEGFR-1 suppression after injury reveals a novel antiangiogenic activity of VEGF-A. J. Clin. Invest. 116: 422-429, 2006.
18. Patten, I. S., Rana, S., Shahul, S., Rowe, G. C., Jang, C., Liu, L., Hacker, M. R., Rhee, J. S., Mitchell, J., Mahmood, F., Hess, P., Farrell, C., and 9 others. Cardiac angiogenic imbalance leads to peripartum cardiomyopathy. Nature 485: 333-338, 2012.
19. Satoh, H., Yoshida, M. C., Matsushime, H., Shibuya, M., Sasaki, M. Regional localization of the human c-ros-1 on 6q22 and fit on 13q12. Jpn. J. Cancer Res. 78: 772-775, 1987.
20. Sela, S., Itin, A., Natanson-Yaron, S., Greenfield, C., Goldman-Wohl, D., Yagel, S., Keshet, E. A novel human-specific soluble vascular endothelial growth factor receptor 1: cell type-specific splicing and implications to vascular endothelial growth factor homeostasis and preeclampsia. Circ. Res. 102: 1566-1574, 2008.
21. Shalaby, F., Rossant, J., Yamaguchi, T. P., Gertsenstein, M., Wu, X.-F., Breitman, M. L., Schuh, A. C. Failure of blood-island formation and vasculogenesis in Flk-l-deficient mice. Nature 376: 62-65, 1995.
22. Shibuya, M., Yamaguchi, S., Yamane, A., Ikeda, T., Tojo, A., Matsushime, H., Sato, M. Nucleotide sequence and expression of a novel human receptor-type tyrosine-kinase gene (flt) closely related to the fms family. Oncogene 5: 519-524, 1990.
23. Stefater, J. A., III, Lewkowich, I., Rao, S., Mariggi, G., Carpenter, A. C., Burr, A. R., Fan, J., Ajima, R., Molkentin, J. D., Williams, B. O., Wills-Karp, M., Pollard, J. W., Yamaguchi, T., Ferrara, N., Gerhardt, H., Lang, R. A. Regulation of angiogenesis by a noncanonical Wnt-Flt1 pathway in myeloid cells. Nature 474: 511-515, 2011.
24. Verma, M., Asakura, Y., Hirai, H., Watanabe, S., Tastad, C., Fong, G.-H., Ema, M., Call, J. A., Lowe, D. A., Asakura, A. Flt-1 haploinsufficiency ameliorates muscular dystrophy phenotype by developmentally increased vasculature in mdx mice. Hum. Molec. Genet. 19: 4145-4159, 2010.
25. Wiesmann, C., Fuh, G., Christinger, H. W., Eigenbrot, C., Wells, J. A., de Vos, A. M. Crystal structure at 1.7 Angstrom resolution of VEGF in complex with domain 2 of the Flt-1 receptor. Cell 91: 695-704, 1997.
26. Wulff, C., Wilson, H., Rudge, J. S., Wiegand, S. J., Lunn, S. F., Fraser, H. M. Luteal angiogenesis: prevention and intervention by treatment with vascular endothelial growth factor trapA40. J. Clin. Endocr. Metab. 86: 3377-3386, 2001.
27. Yoshida, M. C., Satoh, H., Matsushime, H., Shibuya, M., Sasaki, M. Two ros-related protooncogenes, c-ros-1 and flt, are regionally mapped on human chromosomes 6q22 and 13q12, respectively. (Abstract) Cytogenet. Cell Genet. 46: 724 only, 1987.
28. Zhang, F., Zarkada, G., Han, J., Li, J., Dubrac, A., Ola, R., Genet, G., Boye, K., Michon, P., Kunzel, S. E., Camporez, J. P., Singh, A. K., Fong, G.-H., Simons, M., Tso, P., Fernandez-Hernando C., Shulman, G. I., Sessa, W. C., Eichmann, A. Lacteal junction zippering protects against diet-induced obesity. Science 361: 599-603, 2018.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. A Flt-1 decoy comprising:
(a) a VEGF binding domain of Flt-1; and
(b) a membrane anchoring domain;
in which the Flt-1 decoy does not substantially comprise an intracellular domain.

2. A Flt-1 decoy according to claim 1 which is not soluble.

3. A Flt-1 decoy according to claim 1, in which the Flt-1 decoy is not capable of signal transduction.

4. A Flt-1 decoy according to claim 1, in which the Flt-1 decoy lacks an intracellular signalling domain of Flt-1.

5. A Flt-1 decoy according to claim 1, in which the VEGF binding domain of Flt-1 comprises a sequence comprising amino acids 27 to 250 of GenBank Accession Number: NP_002010.2 or a fragment, homologue, variant or derivative thereof capable of binding VEGF, preferably VEGF-A (NP_001020537.2) or VEGF-B (NP_001230662.1).

6. A Flt-1 decoy according to claim 1, in which the membrane anchoring domain comprises a transmembrane domain of Flt1 comprising amino acids 759 to 780 of GenBank Accession Number: NP_002010.2 (SEQ ID NO: 5) or a fragment, homologue, variant or derivative thereof capable of anchoring the Flt-1 decoy to a cell membrane.

7. A Flt-1 decoy according to claim 1 which comprises the sequence of Flt-1 (GenBank Accession Number: NP_002010.2) but without the intracellular domain (amino acids 819 to 1338 of GenBank Accession Number: NP_002010.2) or a fragment, homologue, variant or derivative thereof which is capable of binding VEGF and which is not soluble.

8. A combination of a Flt-1 decoy according to claim 1 together with a mirtron capable of inhibiting VEGF.

9. A combination according to claim 8, in which the mirtron comprises:
(a)
(SEQ ID NO: 1)
GTAAATGTATGTATGTGGGTGTTCAAGAGACACCCACACACATACATC
TCAGTTTTTTCTCTTTCTTTCAG;
or
(b)
(SEQ ID NO: 2)
GTAGATTATGCGGATTAAATTTCAAGAGAGTTTGATCCGCATAATCTGT
CAGTTTTTTCTCTTTCTTTCAG.

10. A method of inhibiting VEGF comprising administering to a subject the Flt-1 decoy according to claim 1.

11. A method of treatment of a disease associated with VEGF expression comprising administering to a subject the Flt-1 decoy according to claim 1.

12. The method of treatment of a disease associated with VEGF expression according to claim 11, wherein the disease comprises macular degeneration, age related macular degeneration (AMD) or wet AMD, corneal neovascularization, diabetic retinopathy, retinal vein occlusions, retinopathy of prematurity, an ocular disease presenting with neovascularization, colon cancer, lung cancer, breast cancer, gastrointestinal stromal cancer, liver cancer, ovarian cancer, fallopian tube cancer, cervical cancer, primary peritoneal cancer, thyroid cancer, pancreatic neuroendocrine tumour, soft tissue sarcoma, glioblastoma or renal-cell carcinoma.

13. A nucleic acid encoding a Flt-1 decoy according to claim 1.

14. An expression vector encoding a Flt-1 decoy according to claim 1 and a mirtron-capable of inhibiting VEGF.

15. An expression vector according to claim 14, wherein the expression vector is a viral expression vector, an adenoviral expression vector, an adeno-associated expression vector, a plasmid construct naked or complexed with liposomes or polymersomes or a dumbbell DNA construct.

16. The expression vector of claim 14, wherein the mitron comprises:
(a)
(SEQ ID NO: 1)
GTAAATGTATGTATGTGGGTGTTCAAGAGACACCCACACACATACATC
TCAGTTTTTTCTCTTTCTTTCAG;
or
(b)
(SEQ ID NO: 2)
GTAGATTATGCGGATTAAATTTCAAGAGAGTTTGATCCGCATAATCTGT
CAGTTTTTTCTCTTTCTTTCAG.

17. The Flt-1 decoy according to claim 3, wherein the Flt-1 decoy is not capable of signal transduction through an SHC-GRB2, PI3K or PLC pathway.

18. The Flt-1 decoy according to claim 4, wherein the Flt-1 decoy lacks an intracellular signalling domain of Flt-1 comprising amino acids 819 to 1338 of GenBank Accession Number: NP_002010.2.


SEQUENCE LISTING (25 sequences)

SEQ ID NO: 1 - DNA, 71 bp, Artificial Sequence
  Mirtron
gtaaatgtatgtatgtgggtgttcaagagacacccacacacatacatctcagttttttctctttctttcag

SEQ ID NO: 2 - DNA, 71 bp, Artificial Sequence
  Mirtron
gtagattatgcggattaaatttcaagagagtttgatccgcataatctgtcagttttttctctttctttcag

SEQ ID NO: 3 - protein, 412 aa, Artificial Sequence
  GenBank Accession Number NP_001020537.2
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 4 - protein, 250 aa, Artificial Sequence
  GenBank Accession Number NP_002010.2 1-250
MVSYWDTGVLLCALLSCLLLTGSSSGSKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVL

SEQ ID NO: 5 - protein, 22 aa, Artificial Sequence
  GenBank Accession Number NP_002010.2 (residues 759-780)
LITLTCTCVAATLFWLLLTLFI

SEQ ID NO: 6 - protein, 2454 aa, Artificial Sequence
  Flt-1 Decoy
ATGGTTTCTTACTGGGACACGGGGGTACTCCTTTGCGCACTTCTGTCTTGTTTGCTTCTGACGGGGTCCTCTTCTGGGTCTAAGCTGAAAGATCCGGAGCTGTCTCTGAAGGGCACGCAACACATCATGCAAGCTGGTCAAACTTTGCATCTCCAGTGCCGGGGGGAGGCTGCGCATAAGTGGTCTTTGCCTGAAATGGTATCAAAGGAGTCCGAACGCCTCAGTATAACTAAAAGCGCATGTGGTCGGAACGGAAAACAATTTTGTAGCACGCTCACACTCAATACTGCTCAAGCCAATCATACCGGATTTTACTCTTGCAAATATCTTGCGGTACCGACCTCTAAAAAGAAAGAAACCGAAAGCGCCATCTACATCTTCATTTCTGACACAGGCCGGCCATTTGTTGAAATGTATTCAGAGATCCCTGAAATCATCCACATGACTGAGGGGCGAGAACTTGTTATACCCTGCCGGGTCACCAGTCCCAACATTACGGTGACCCTCAAAAAATTCCCACTGGATACGCTTATCCCGGACGGGAAGCGCATTATATGGGACTCTCGAAAAGGGTTTATCATTTCAAATGCCACGTACAAAGAAATCGGTCTGCTGACCTGCGAGGCCACGGTGAATGGCCACTTGTATAAGACTAATTACCTCACTCACCGCCAGACAAACACAATTATCGATGTACAGATCTCCACACCTCGACCCGTTAAGCTGCTCAGAGGGCATACACTTGTACTTAACTGCACAGCCACCACCCCGCTGAATACGAGAGTACAGATGACCTGGTCATATCCGGACGAGAAGAACAAAAGAGCTTCCGTGCGAAGGCGAATCGACCAGTCAAACTCCCATGCCAACATTTTCTACTCTGTTCTGACGATCGACAAAATGCAGAACAAAGATAAGGGTTTGTACACTTGTCGAGTCCGGAGTGGTCCATCTTTCAAAAGTGTAAATACTTCAGTGCATATCTACGATAAAGCCTTTATTACGGTCAAGCATCGAAAGCAACAAGTACTCGAAACTGTAGCAGGGAAACGCTCCTACCGGTTGTCTATGAAGGTAAAGGCTTTTCCCAGCCCCGAAGTAGTGTGGCTCAAAGATGGGCTTCCGGCGACGGAGAAGAGCGCTAGGTACTTGACAAGGGGCTACTCACTCATAATAAAGGACGTGACGGAAGAGGACGCGGGGAATTATACAATACTTTTGTCCATAAAACAATCTAACGTTTTCAAAAACCTCACGGCGACTTTGATTGTCAATGTGAAACCTCAAATCTACGAGAAAGCTGTCTCTTCCTTCCCGGACCCAGCGTTGTATCCACTCGGATCTAGGCAGATTCTCACCTGTACAGCCTACGGGATACCGCAGCCTACTATTAAATGGTTTTGGCACCCATGTAACCATAACCACTCAGAAGCTCGCTGCGATTTCTGCTCTAATAATGAAGAGAGTTTCATACTTGACGCGGATTCCAACATGGGAAACCGCATTGAGTCAATTACCCAACGGATGGCAATCATCGAAGGGAAGAACAAGATGGCCTCAACTCTCGTGGTAGCAGATAGCCGAATTTCAGGAATATACATTTGTATCGCGTCTAATAAGGTAGGAACTGTCGGCCGAAATATATCCTTTTACATCACGGATGTCCCCAACGGATTTCATGTAAATCTGGAAAAGATGCCCACAGAAGGAGAGGATCTGAAACTTTCCTGTACGGTAAATAAGTTCCTCTATCGCGACGTAACATGGATTTTGCTCCGGACCGTTAACAACCGCACCATGCATTACAGTATATCTAAGCAGAAGATGGCCATTACTAAAGAGCATTCTATTACACTGAACCTCACTATCATGAATGTATCTCTTCAGGATAGTGGCACGTACGCGTGTCGCGCTAGGAATGTGTATACTGGCGAGGAAATACTCCAGAAGAAAGAGATTACGATCAGGGACCAGGAGGCACCATACCTCCTGAGAAACCTTTCTGACCACACGGTGGCCATAAGTAGTAGTACGACACTTGATTGCCATGCGAACGGTGTTCCGGAACCACAGATCACATGGTTTAAGAACAATCACAAAATCCAGCAGGAGCCCGGGATCATACTTGGACCTGGGAGCTCCACGTTGTTTATTGAAAGGGTTACCGAAGAGGACGAAGGGGTCTATCATTGTAAGGCAACAAATCAAAAGGGATCAGTTGAAAGTAGTGCATACTTGACCGTGCAAGGAACTAGTGATAAATCCAACCTTGAGCTGATTACGTTGACGTGCACGTGCGTAGCAGCTACCTTGTTCTGGCTGCTCTTGACCCTGTTCATTCGGAAAATGAAAAGGTCCTCTAGTGAGATAAAAACTGACTACCTTTCCATAATAATGGACCCGGACGAAGTTCCACTGGACGAACAGTGTGAACGCCTGCCGTACGACGCGTCC

SEQ ID NO: 7 - DNA, 35 bp, Artificial Sequence
  Synthetic Primer
acagctagcaccatggtttcttactgggacacggg

SEQ ID NO: 8 - DNA, 36 bp, Artificial Sequence
  Synthetic Primer
atcggatcctcattaagcgtagtctggaacgtcata

SEQ ID NO: 9 - DNA, 2502 bp, Artificial Sequence
  coFlt1
gctagcaccatggtttcttactgggacacgggggtactcctttgcgcacttctgtcttgtttgcttctgacggggtcctcttctgggtctaagctgaaagatccggagctgtctctgaagggcacgcaacacatcatgcaagctggtcaaactttgcatctccagtgccggggggaggctgcgcataagtggtctttgcctgaaatggtatcaaaggagtccgaacgcctcagtataactaaaagcgcatgtggtcggaacggaaaacaattttgtagcacgctcacactcaatactgctcaagccaatcataccggattttactcttgcaaatatcttgcggtaccgacctctaaaaagaaagaaaccgaaagcgccatctacatcttcatttctgacacaggccggccatttgttgaaatgtattcagagatccctgaaatcatccacatgactgaggggcgagaacttgttataccctgccgggtcaccagtcccaacattacggtgaccctcaaaaaattcccactggatacgcttatcccggacgggaagcgcattatatgggactctcgaaaagggtttatcatttcaaatgccacgtacaaagaaatcggtctgctgacctgcgaggccacggtgaatggccacttgtataagactaattacctcactcaccgccagacaaacacaattatcgatgtacagatctccacacctcgacccgttaagctgctcagagggcatacacttgtacttaactgcacagccaccaccccgctgaatacgagagtacagatgacctggtcatatccggacgagaagaacaaaagagcttccgtgcgaaggcgaatcgaccagtcaaactcccatgccaacattttctactctgttctgacgatcgacaaaatgcagaacaaagataagggtttgtacacttgtcgagtccggagtggtccatctttcaaaagtgtaaatacttcagtgcatatctacgataaagcctttattacggtcaagcatcgaaagcaacaagtactcgaaactgtagcagggaaacgctcctaccggttgtctatgaaggtaaaggcttttcccagccccgaagtagtgtggctcaaagatgggcttccggcgacggagaagagcgctaggtacttgacaaggggctactcactcataataaaggacgtgacggaagaggacgcggggaattatacaatacttttgtccataaaacaatctaacgttttcaaaaacctcacggcgactttgattgtcaatgtgaaacctcaaatctacgagaaagctgtctcttccttcccggacccagcgttgtatccactcggatctaggcagattctcacctgtacagcctacgggataccgcagcctactattaaatggttttggcacccatgtaaccataaccactcagaagctcgctgcgatttctgctctaataatgaagagagtttcatacttgacgcggattccaacatgggaaaccgcattgagtcaattacccaacggatggcaatcatcgaagggaagaacaagatggcctcaactctcgtggtagcagatagccgaatttcaggaatatacatttgtatcgcgtctaataaggtaggaactgtcggccgaaatatatccttttacatcacggatgtccccaacggatttcatgtaaatctggaaaagatgcccacagaaggagaggatctgaaactttcctgtacggtaaataagttcctctatcgcgacgtaacatggattttgctccggaccgttaacaaccgcaccatgcattacagtatatctaagcagaagatggccattactaaagagcattctattacactgaacctcactatcatgaatgtatctcttcaggatagtggcacgtacgcgtgtcgcgctaggaatgtgtatactggcgaggaaatactccagaagaaagagattacgatcagggaccaggaggcaccatacctcctgagaaacctttctgaccacacggtggccataagtagtagtacgacacttgattgccatgcgaacggtgttccggaaccacagatcacatggtttaagaacaatcacaaaatccagcaggagcccgggatcatacttggacctgggagctccacgttgtttattgaaagggttaccgaagaggacgaaggggtctatcattgtaaggcaacaaatcaaaagggatcagttgaaagtagtgcatacttgaccgtgcaaggaactagtgataaatccaaccttgagctgattacgttgacgtgcacgtgcgtagcagctaccttgttctggctgctcttgaccctgttcattcggaaaatgaaaaggtcctctagtgagataaaaactgactacctttccataataatggacccggacgaagttccactggacgaacagtgtgaacgcctgccgtacgacgcgtcctatccttatgacgttccagactacgcttaatgaggatcc

SEQ ID NO: 10 - DNA, 57 bp, Artificial Sequence
  Synthetic Primer
acaggatcctagtgatggtggtgatgatggcctcccttaacgggtcgaggtgtggag

SEQ ID NO: 11 - DNA, 23 bp, Artificial Sequence
  Synthetic Primer
gcgtcctatccttatgacgttcc

SEQ ID NO: 12 - DNA, 764 bp, Artificial Sequence
  scoFlt1
gctagcaccatggtttcttactgggacacgggggtactcctttgcgcacttctgtcttgtttgcttctgacggggtcctcttctgggtctaagctgaaagatccggagctgtctctgaagggcacgcaacacatcatgcaagctggtcaaactttgcatctccagtgccggggggaggctgcgcataagtggtctttgcctgaaatggtatcaaaggagtccgaacgcctcagtataactaaaagcgcatgtggtcggaacggaaaacaattttgtagcacgctcacactcaatactgctcaagccaatcataccggattttactcttgcaaatatcttgcggtaccgacctctaaaaagaaagaaaccgaaagcgccatctacatcttcatttctgacacaggccggccatttgttgaaatgtattcagagatccctgaaatcatccacatgactgaggggcgagaacttgttataccctgccgggtcaccagtcccaacattacggtgaccctcaaaaaattcccactggatacgcttatcccggacgggaagcgcattatatgggactctcgaaaagggtttatcatttcaaatgccacgtacaaagaaatcggtctgctgacctgcgaggccacggtgaatggccacttgtataagactaattacctcactcaccgccagacaaacacaattatcgatgtacagatctccacacctcgacccgttaagggaggccatcatcaccaccatcactaggatcc

SEQ ID NO: 13 - DNA, 2644 bp, Artificial Sequence
  coFlt1-Mirt
gctagcaccatggtttcttactgggacacgggggtactcctttgcgcacttctgtcttgtttgcttctgacggggtcctcttctgggtctaagctgaaagatccggagctgtctctgaaggtaaatgtatgtatgtgggtgttcaagagacacccacacacatacatctcagttttttctctttctttcagggcacgcaacacatcatgcaagctggtcaaactttgcatctccagtgccggggggaggctgcgcataagtggtctttgcctgaaatggtatcaaaggtagattatgcggattaaatttcaagagagtttgatccgcataatctgtcagttttttctctttctttcaggagtccgaacgcctcagtataactaaaagcgcatgtggtcggaacggaaaacaattttgtagcacgctcacactcaatactgctcaagccaatcataccggattttactcttgcaaatatcttgcggtaccgacctctaaaaagaaagaaaccgaaagcgccatctacatcttcatttctgacacaggccggccatttgttgaaatgtattcagagatccctgaaatcatccacatgactgaggggcgagaacttgttataccctgccgggtcaccagtcccaacattacggtgaccctcaaaaaattcccactggatacgcttatcccggacgggaagcgcattatatgggactctcgaaaagggtttatcatttcaaatgccacgtacaaagaaatcggtctgctgacctgcgaggccacggtgaatggccacttgtataagactaattacctcactcaccgccagacaaacacaattatcgatgtacagatctccacacctcgacccgttaagctgctcagagggcatacacttgtacttaactgcacagccaccaccccgctgaatacgagagtacagatgacctggtcatatccggacgagaagaacaaaagagcttccgtgcgaaggcgaatcgaccagtcaaactcccatgccaacattttctactctgttctgacgatcgacaaaatgcagaacaaagataagggtttgtacacttgtcgagtccggagtggtccatctttcaaaagtgtaaatacttcagtgcatatctacgataaagcctttattacggtcaagcatcgaaagcaacaagtactcgaaactgtagcagggaaacgctcctaccggttgtctatgaaggtaaaggcttttcccagccccgaagtagtgtggctcaaagatgggcttccggcgacggagaagagcgctaggtacttgacaaggggctactcactcataataaaggacgtgacggaagaggacgcggggaattatacaatacttttgtccataaaacaatctaacgttttcaaaaacctcacggcgactttgattgtcaatgtgaaacctcaaatctacgagaaagctgtctcttccttcccggacccagcgttgtatccactcggatctaggcagattctcacctgtacagcctacgggataccgcagcctactattaaatggttttggcacccatgtaaccataaccactcagaagctcgctgcgatttctgctctaataatgaagagagtttcatacttgacgcggattccaacatgggaaaccgcattgagtcaattacccaacggatggcaatcatcgaagggaagaacaagatggcctcaactctcgtggtagcagatagccgaatttcaggaatatacatttgtatcgcgtctaataaggtaggaactgtcggccgaaatatatccttttacatcacggatgtccccaacggatttcatgtaaatctggaaaagatgcccacagaaggagaggatctgaaactttcctgtacggtaaataagttcctctatcgcgacgtaacatggattttgctccggaccgttaacaaccgcaccatgcattacagtatatctaagcagaagatggccattactaaagagcattctattacactgaacctcactatcatgaatgtatctcttcaggatagtggcacgtacgcgtgtcgcgctaggaatgtgtatactggcgaggaaatactccagaagaaagagattacgatcagggaccaggaggcaccatacctcctgagaaacctttctgaccacacggtggccataagtagtagtacgacacttgattgccatgcgaacggtgttccggaaccacagatcacatggtttaagaacaatcacaaaatccagcaggagcccgggatcatacttggacctgggagctccacgttgtttattgaaagggttaccgaagaggacgaaggggtctatcattgtaaggcaacaaatcaaaagggatcagttgaaagtagtgcatacttgaccgtgcaaggaactagtgataaatccaaccttgagctgattacgttgacgtgcacgtgcgtagcagctaccttgttctggctgctcttgaccctgttcattcggaaaatgaaaaggtcctctagtgagataaaaactgactacctttccataataatggacccggacgaagttccactggacgaacagtgtgaacgcctgccgtacgacgcgtcctatccttatgacgttccagactacgcttaatgaggatcc

SEQ ID NO: 14 - DNA, 35 bp, Artificial Sequence; identical to SEQ ID NO: 7
  Primer NheI coFlt1 F

SEQ ID NO: 15 - DNA, 36 bp, Artificial Sequence; identical to SEQ ID NO: 8
  Primer BamHI coFlt1 R

SEQ ID NO: 16 - DNA, 23 bp, Artificial Sequence
  Primer sco-Flt1 F
gcgtcctatccttatgacgttcc

SEQ ID NO: 17 - DNA, 57 bp, Artificial Sequence; identical to SEQ ID NO: 10
  Primer BamHI sco-Flt1-His R

SEQ ID NO: 18 - DNA, 66 bp, Artificial Sequence
  Primer Bbs1 CoFlt1 Mirt 1 R
tatgaagactaggtgtctcttgaacacccacatacatacatttaccttcagagacagctccggatc

SEQ ID NO: 19 - DNA, 75 bp, Artificial Sequence
  Primer Bbs1 CoFlt1 Mirt 1 F
tatgaagactacacccacacacatacatctcagttttttctctttctttcagggcacgcaacacatcatggtatc

SEQ ID NO: 20 - DNA, 60 bp, Artificial Sequence
  Primer BbsI CoFlt1 Mirt 8 R
tgaagactacttgaaatttaatccgcataatctacctttgataccatttcaggcaaagac

SEQ ID NO: 21 - DNA, 81 bp, Artificial Sequence
  Primer BbsI CoFlt1 Mirt 8 F
atgaagactacaagagagtttgatccgcataatctgtcagttttttctctttctttcaggagtccgaacgcctcagtataa

SEQ ID NO: 22 - DNA, 21 bp, Artificial Sequence
  Primer VEGFA qPCR F
cggatcaaacctcaccaaggc

SEQ ID NO: 23 - DNA, 19 bp, Artificial Sequence
  Primer VEGFA qPCR R
agggaggctccttcctcct

SEQ ID NO: 24 - DNA, 20 bp, Artificial Sequence
  Primer GAPDH qPCR F
aaggtgaaggtcggagtcaa

SEQ ID NO: 25 - DNA, 20 bp, Artificial Sequence
  Primer GAPDH qPCR R
gaagatggtgatgggatttc